(19) United States
(12) Patent Application Publication
(10) Pub. No.: US 2024/0423815 A1
(43) Pub. Date: Dec. 26, 2024

(54) ACTUATOR AND PROSTHESIS

(71) Applicant: Sony Group Corporation, Tokyo (JP)

(72) Inventors: Ken ENDO, Tokyo (JP); Ryusuke MORITA, Tokyo (JP); Masato TANAKA, Tokyo (JP)

(73) Assignee: Sony Group Corporation, Tokyo (JP)

(21) Appl. No.: 18/693,673

(22) PCT Filed: Mar. 11, 2022
(86) PCT No.: PCT/JP2022/010846

§ 371 Date: Mar. 20, 2024

(30) Foreign Application Priority Data
Sep. 27, 2021 (JP) 2021-156632

(51) Int. Cl. A61F 2/64; A61F 2/50; A61F 2/70

(52) U.S. Cl. CPC A61F 2/64; A61F 2/70; A61F 2002/5079

(57) ABSTRACT

The present disclosure relates to an actuator and a prosthesis that can propose an actuator and a prosthesis that can downsize a device.

An actuator includes: a leaf spring that includes a bent portion bent in a U shape and a first extending portion extending from the bent portion, a first one end portion that is an end portion of the first extending portion on a side of the bent portion being cantilevered, the first extending portion being bendable and deformable in a plate thickness direction according to torque by transmitting the torque; and a first support member that supports a part of the first extending portion on a bending direction side in a case where torque transmitted in a first direction by the first extending portion is larger than a predetermined value. The present technology can be applied to, for example, a prosthesis.

### TECHNICAL FIELD

The present disclosure relates to an actuator and a prosthesis, and more particularly to an actuator and a prosthesis using a leaf spring.

### BACKGROUND ART

Conventionally, a prosthesis using a series elastic actuator (SEA) using a leaf spring as an elastic member has been proposed (see, for example, Patent Document 1).

### CITATION LIST

### Patent Document

Patent Document 1: WO 2017/212708

### SUMMARY OF THE INVENTION

### Problems to be Solved by the Invention

Meanwhile, in the fields related to robots, it is desired to further downsize devices such as the actuator and the prosthesis described in Patent Document 1.

The present technology has been made in view of such a situation, and proposes an actuator and a prosthesis capable of downsizing a device.

### Solutions to Problems

An actuator according to a first aspect of the present disclosure includes: a leaf spring that includes a bent portion bent in a U shape and a first extending portion extending from the bent portion, a first one end portion that is an end portion of the first extending portion on a side of the bent portion being cantilevered, the first extending portion being bendable and deformable in a plate thickness direction according to torque by transmitting the torque; and a first support member that supports a part of the first extending portion on a bending direction side in a case where torque transmitted in a first direction by the first extending portion is larger than a predetermined value.

In the first aspect of the present disclosure, in a case where the torque transmitted in the first direction by the first extending portion of the leaf spring is larger than a predetermined value, a part of the first extending portion is supported on the bending direction side.

A prosthesis according to a second aspect of the present disclosure includes: a thigh-side member; a lower-leg-side member; and an actuator that connects the thigh-side member and the lower-leg-side member, and transmits torque to one of the thigh-side member and the lower-leg-side member to rotate one of the thigh-side member and the lower-leg-side member relative to the other, in which the actuator includes: a leaf spring including a bent portion bent in a U shape and an extending portion extending from the bent portion, one end portion which is an end portion of the extending portion on a side of the bent portion being cantilevered, the extending portion being bendable and deformable in a plate thickness direction according to torque by transmitting the torque; and a support member that supports a part of the extending portion on a bending direction side in a case where torque transmitted by the extending portion is larger than a predetermined value.

In the second aspect of the present disclosure, the thigh-side member and the lower-leg-side member are connected, and torque is transmitted to one of the thigh-side member and the lower-leg-side member, whereby the one of the thigh-side member and the lower-leg-side member is relatively rotated with respect to the other, and in a case where the torque transmitted by the extending portion of the leaf spring is larger than a predetermined value, a part of the extending portion is supported on the bending direction side.

### BRIEF DESCRIPTION OF DRAWINGS

FIG. 1 is a schematic diagram illustrating an example of a schematic configuration of a prosthesis according to an embodiment of the present disclosure.

FIG. 2 is a perspective view of a thigh-side member of the prosthesis according to the embodiment.

FIG. 3 is a perspective view of a thigh-side member of the prosthesis according to the embodiment.

FIG. 4 is a cross-sectional view of a thigh-side member of the prosthesis according to the embodiment.

FIG. 5 is an explanatory diagram for explaining a transmission path of power and a signal in the prosthesis according to the embodiment.

FIG. 6 is an explanatory diagram illustrating a state of deformation of the leaf spring in a state where a portion on one end portion side of the leaf spring does not abut on the deformation guide.

FIG. 7 is a shearing force diagram of the leaf spring in the state illustrated in FIG. 6.

FIG. 8 is a bending moment diagram of the leaf spring in the state illustrated in FIG. 6.

FIG. 9 is an explanatory diagram for explaining a relationship between a deflection angle θ and a distance s.

FIG. 10 is an explanatory diagram illustrating a state of deformation of the leaf spring in a state where a portion on one end portion side of the leaf spring abuts on the deformation guide.

FIG. 11 is an explanatory diagram for explaining deformation on the other end portion side of the leaf spring in a state where a portion on one end portion side of the leaf spring abuts on the deformation guide.

FIG. 12 is a shearing force diagram of the leaf spring in the state illustrated in FIG. 10.

FIG. 13 is a bending moment diagram of the leaf spring in the state illustrated in FIG. 10.

FIG. 14 is an explanatory diagram illustrating an example of a relationship between an other end portion deflection angle θ_{L} of the leaf spring and a load P applied to the leaf spring.

FIG. 15 is an explanatory diagram illustrating an example of a relationship between an other end portion deflection angle θ_{L} of the leaf spring and rigidity.

FIG. 16 is an explanatory diagram illustrating an example of a relationship between an other end portion deflection angle θ_{L} of the leaf spring and a load change rate.

FIG. 17 is an explanatory diagram illustrating an example of a relationship between a load P applied to a leaf spring and a load change rate.

FIG. 18 is a schematic diagram illustrating a first embodiment of the actuator according to the embodiment of the present disclosure.

FIG. 19 is a schematic diagram illustrating an example of a state of an actuator in a case where one end portion of a leaf spring relatively stops with respect to a rotating body and in a case where the leaf spring rotates.

FIG. 20 is a schematic diagram illustrating a second embodiment of the torque sensor according to the embodiment of the present disclosure.

### MODE FOR CARRYING OUT THE INVENTION

Hereinafter, preferred embodiments of the present disclosure will be described in detail with reference to the accompanying drawings. Note that, in the present specification and drawings, components having substantially the same functional configuration are denoted by the same reference signs, and redundant description is omitted.

Note that the description will be given in the following order.
1. Prosthesis According to Embodiment of Present Disclosure
2. Overview of Present Technology
   2-1. Deformation of Leaf Spring
   2-2. Mechanical Characteristics of Leaf Spring
3. First Embodiment of Actuator
4. Second Embodiment of Actuator
5. Modifications
6. Others

### 1. Prosthesis According to Embodiment of Present Disclosure

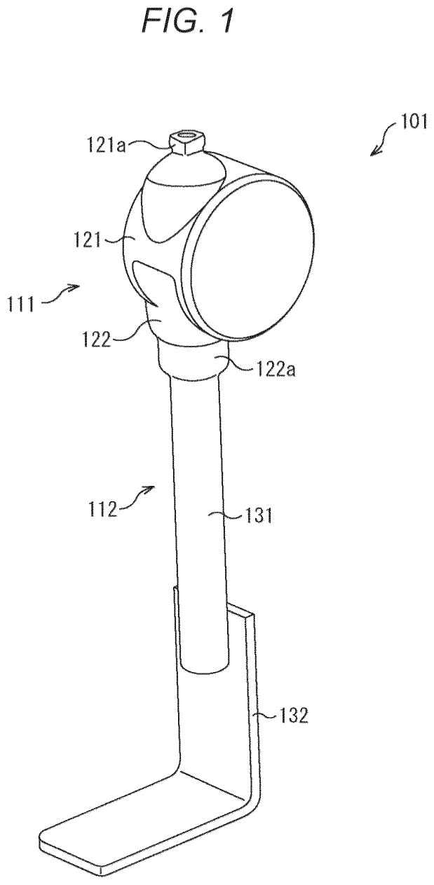

First, a prosthesis 101 according to an embodiment of the present disclosure will be described with reference to FIGS. 1 to 5.

FIG. 1 is a schematic diagram illustrating an example of a schematic configuration of a prosthesis 101.

The prosthesis 101 is worn by a user who is a wearer, and is used to support the weight of the user.

The prosthesis 101 includes a thigh-side member 111 and a lower-leg-side member 112. The thigh-side member 111 includes a main body portion 121 and a knee joint 122. The lower-leg-side member 112 includes an extending portion 131 and a ground contact portion 132.

The main body portion 121 has a substantially columnar shape. A connection portion 121a at the upper end of the main body portion 121 is connected to a socket member (not illustrated) that accommodates a portion of the user to which the prosthesis 101 is to be attached. In a state where the prosthesis 101 is worn by the user, the connection portion 121a is located on the upper side in the vertical direction in the thigh-side member 111, and the above-described socket member opens vertically upward. An internal space is formed inside the main body portion 121. As described later, various members are accommodated in the internal space of the main body portion 121.

The knee joint 122 is connected to the main body portion 121 so as to be rotatable relative to the main body portion 121. The rotation direction of the knee joint 122 is guided by the main body portion 121. Specifically, the knee joint 122 is connected to the main body portion 121 so as to be rotatable about the central axis of the main body portion 121.

The extending portion 131 of the lower-leg-side member 112 is a substantially columnar member, and an upper end thereof is connected to a connection portion 122a of the knee joint 122. The ground contact portion 132 has a shape bent substantially at a right angle, a lower end side of the extending portion 131 is connected to a portion extending in the vertical direction, and a portion extending in the horizontal direction abuts on the floor.

The extending portion 131 and the ground contact portion 132 rotate about the central axis of the main body portion 121 as the knee joint 122 of the thigh-side member 111 rotates about the central axis of the main body portion 121. That is, the lower-leg-side member 112 is rotatable relative to the thigh-side member 111.

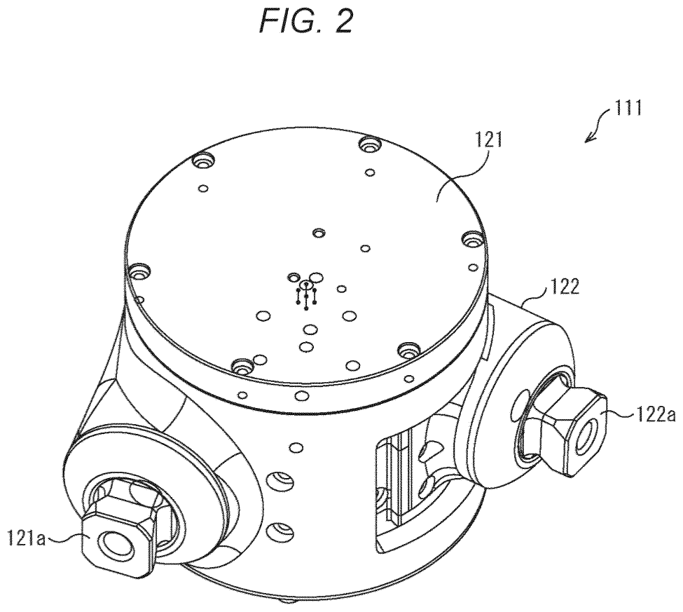

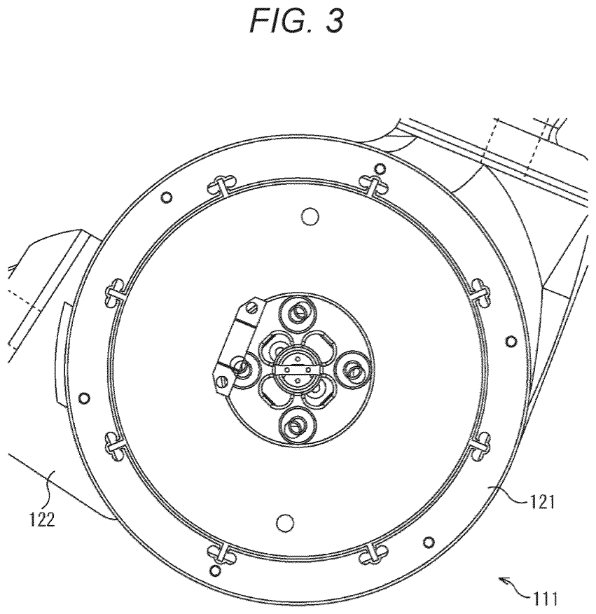

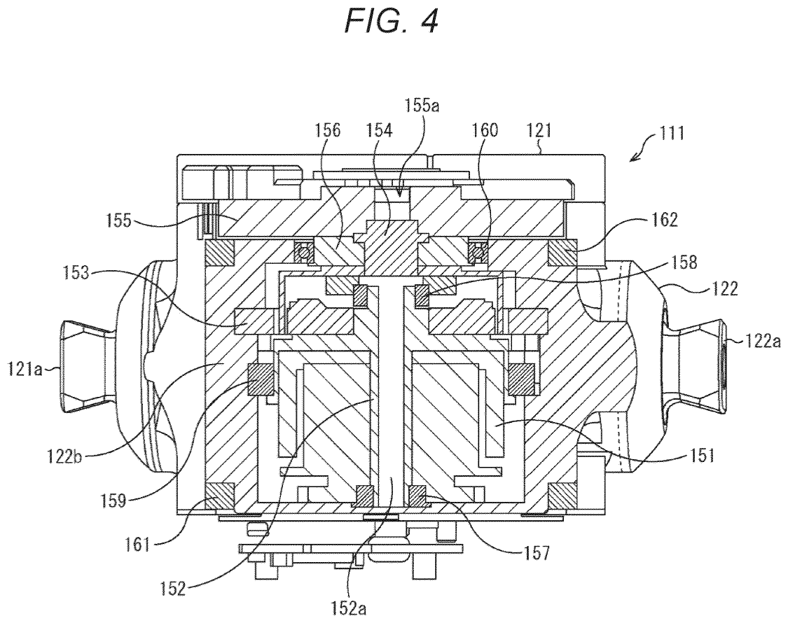

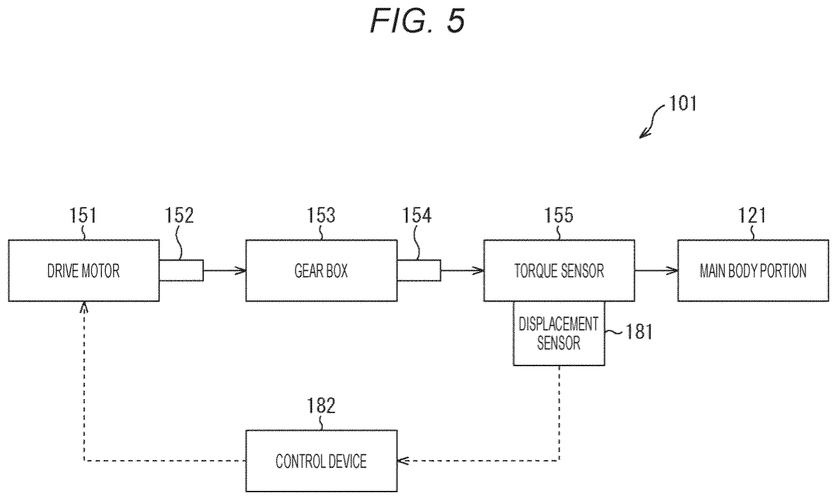

Next, a configuration example of the thigh-side member 111 will be described with reference to FIGS. 2 to 5. FIG. 2 is a perspective view of the thigh-side member 111. FIG. 3 is a perspective view of the thigh-side member 111 in a case where viewed from a surface opposite to FIG. 2. FIG. 4 is a cross-sectional view of the thigh-side member 111. FIG. 5 is an explanatory diagram for explaining a power and signal transmission path in the thigh-side member 111. Note that, in FIG. 5, solid arrows and broken arrows indicate power and signal transmission paths, respectively.

As illustrated in FIG. 4, an internal space is formed inside the main body portion 121. A part of the knee joint 122 (hereinafter, referred to as a housing portion 122b) and a torque sensor 155 are accommodated in the internal space of the main body portion 121. In addition, the connection portion 122a of the knee joint 122 protrudes from the right side of the main body portion 121 in the drawing.

An internal space is formed inside the housing portion 122b of the knee joint 122. The internal space of the knee joint 122 accommodates a drive motor 151, an output shaft 152, and a gear box 153.

For example, a frameless motor is used as the drive motor 151. The lower surface of the drive motor 151 in the drawing is fixed to the lower surface of the housing portion 122b of the knee joint 122 in the drawing.

For example, a shaft (hollow shaft) having a hollow structure is used as the output shaft 152. That is, a hollow portion 152a penetrating in the axial direction is provided inside the output shaft 152. The output shaft 152 is attached to the drive motor 151 such that the rotation axis substantially coincides with the central axis of the drive motor 151. The lower end portion of the output shaft 152 in the drawing is inserted into a bearing 157. The upper end portion of the output shaft 152 in the drawing is inserted into the gear box 153 and the bearing 158. Between the drive motor 151 and the gear box 153, the output shaft 152 extends in the horizontal direction in the drawing, and the portion extending in the horizontal direction is inserted into the bearing 159.

For example, a harmonic gear is used as the gear box 153. The gear box 153 is disposed such that the central axis substantially coincides with the central axis of the drive motor 151. Therefore, the rotation axis of the output shaft 152 substantially coincides with the central axis of the gear box 153.

As an output shaft 154, for example, a shaft (hollow shaft) having a hollow structure is used similarly to the output shaft 152. That is, a hollow portion (not illustrated) penetrating in the axial direction is provided inside the output shaft 154. The lower end portion of the output shaft 154 in the drawing is connected to the gear box 153 such that the rotation axis coincides with the central axis of the gear box 153. Therefore, the rotation axis of the output shaft 154 substantially coincides with the rotation axis of the output shaft 152. In addition, the output shaft 154 is inserted into an annular connection member 156, and the upper end portion thereof is inserted into a communication hole portion 155a of the torque sensor 155. The communication hole portion 155a communicates from one surface to the other surface along the central axis of the torque sensor 155 at the central portion of the torque sensor 155. The rotation axis of the output shaft 154 substantially coincides with the central axis of the communication hole portion 155a.

The hollow portion 152a of the output shaft 152 and the hollow portion of the output shaft 154 overlap in the axial direction. That is, the hollow portion of the output shaft 152 and the hollow portion of the output shaft 154 are connected in the axial direction. As a result, for example, by passing a cable through the hollow portion 152a of the output shaft 152 and the hollow portion of the output shaft 154, the main body portion 121 can be connected in the vertical direction in the drawing. In addition, for example, the magnet of the encoder can be arranged in the hollow portion 152a of the output shaft 152 and the hollow portion of the output shaft 154.

Thus, the thigh-side member 111 can be downsized. Furthermore, since the cable is accommodated inside the thigh-side member 111 without being exposed to the outside, for example, disconnection of the cable can be prevented or the degree of freedom in design can be increased.

The torque sensor 155 is disposed on the housing portion 122b of the knee joint 122 in the drawing. As will be described later, the torque sensor 155 is provided with a leaf spring extending to the left and right in the drawing, and an end portion of the leaf spring is connected to the main body portion 121.

In the drawing, the connection member 156 is disposed at substantially the same height as the upper surface of the housing portion 122b of the knee joint 122 and is inserted into the bearing 160. The connection member 156 connects the output shaft 154 and the lower surface of the torque sensor 155 in the drawing. Accordingly, the torque sensor 155 is rotatable integrally with the output shaft 154 of the gear box 153.

The lower end of the housing portion 122b of the knee joint 122 in the drawing is inserted into the bearing 161. The upper end of the housing portion 122b of the knee joint 122 in the drawing is inserted into the bearing 162.

Torque generated by the drive motor 151 rotating the output shaft 152 is transmitted to the gear box 153. The gear box 153 converts the torque transmitted from the drive motor 151 at a predetermined reduction ratio and transmits the converted torque to the torque sensor 155 via the output shaft 154. The torque transmitted to the torque sensor 155 is transmitted to the main body portion 121 as a target object on the output side via the leaf spring of the torque sensor 155. As a result, the main body portion 121 rotates relative to the knee joint 122 about the output shaft 154. As a result, the thigh-side member 111 rotates relative to the lower-leg-side member 112 connected to the knee joint 122.

As described above, the main body portion 121, the knee joint 122, the drive motor 151, the output shaft 152, the gear box 153, the output shaft 154, the torque sensor 155, the connection member 156, the bearings 157 to 162, and the like constitute an actuator that drives the knee of the prosthesis 101.

A displacement sensor 181 in FIG. 5 detects a displacement amount of the leaf spring of the torque sensor 155 and outputs a detection result to a control device 182.

The control device 182 controls driving of the drive motor 151 on the basis of a detection result of the displacement sensor 181. Specifically, the control device 182 calculates the load applied to the leaf spring on the basis of the measurement value of the displacement amount of the leaf spring corresponding to the detection result. Then, the control device 182 calculates the torque actually transmitted to the main body portion 121 via the leaf spring on the basis of the calculated value of the load. Then, the control device 182 controls the driving of the drive motor 151 on the basis of the calculated value of the torque such that the torque transmitted to the main body portion 121 approaches the target value.

### 2. Overview of Present Technology

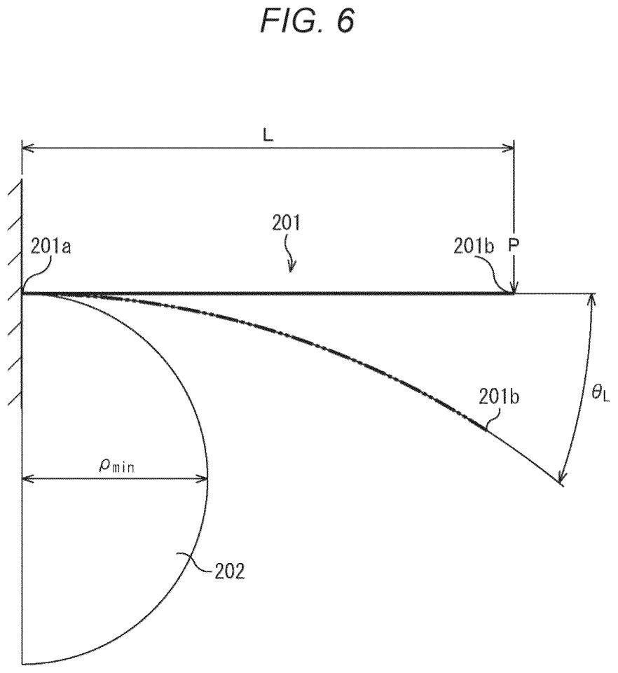

Next, an overview of the present technology will be described prior to the description of the details of the actuator according to the embodiment of the present disclosure with reference to FIGS. 6 to 17. Specifically, matters for facilitating understanding of deformation and mechanical characteristics of the leaf spring in the actuator described later will be described.

### [2-1. Deformation of Leaf Spring]

First, deformation of a leaf spring 201 will be described with reference to FIGS. 6 to 13. Hereinafter, deformation of the leaf spring 201 when the load P is applied in the plate thickness direction to the other end portion 201b of the leaf spring 201 in the system including the leaf spring 201 in which the one end portion 201a is cantilevered and a deformation guide 202 capable of supporting a part of the leaf spring on the bending direction side will be described. The leaf spring 201 is bendable and deformable in the plate thickness direction according to the load P. Further, the deformation guide 202 is provided close to a portion of the one end portion 201a side of the leaf spring 201 in the plate thickness direction of the leaf spring 201, and supports the portion of the one end portion 201a side of the leaf spring 201 on the bending direction side in a case where the load P is larger than a predetermined value.

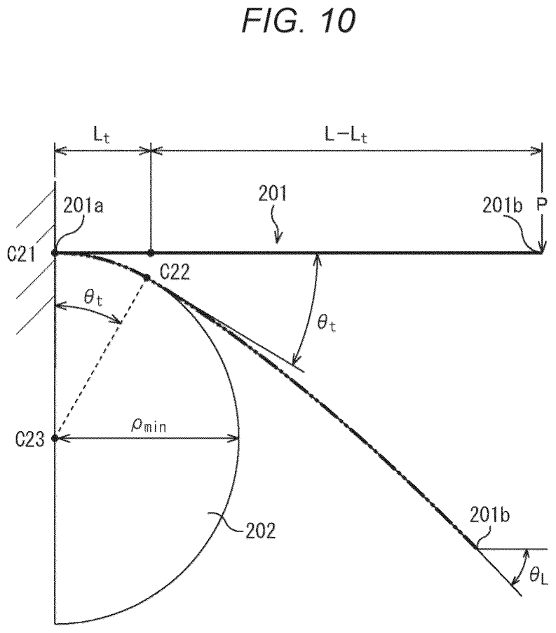

In FIGS. 6 and 10, the leaf spring 201 and the deformation guide 202 are schematically illustrated by a cross-sectional view of a cross section orthogonal to the width direction of the leaf spring 201. In addition, in FIGS. 6 and 10, the neutral axis of the leaf spring 201 is schematically illustrated as the shape of the leaf spring 201. Specifically, a deflection curve indicating the neutral axis of the leaf spring 201 in a case where bending deformation occurs is indicated by a two-dot chain line, and the neutral axis of the leaf spring 201 in a case where bending deformation does not occur is indicated by a solid line. Note that the leaf spring 201 has a rectangular cross-sectional shape. Further, the plate thickness of the leaf spring 201 is denoted by D, the second moment of area is denoted by I, the Young's modulus is denoted by E, and the length in the longitudinal direction is denoted by L.

### (Deformation Guide)

First, the deformation guide 202 will be described. The deformation guide 202 is provided to prevent the leaf spring 201 from being damaged by the applied load P. Specifically, the deformation guide 202 is provided to prevent plastic deformation of the leaf spring 201 due to the load P. The relationship between the bending moment M applied to the leaf spring 201 and the curvature radius ρ of the neutral axis of the leaf spring 201 is expressed by the following Formula (1).

$$\begin{matrix}[ Math . 1 ] & \\ \frac{1}{ρ} = \frac{M}{E I} & ( 1 )\end{matrix}$$

Further, the strain in the longitudinal direction generated in the leaf spring 201 is maximized at the end portion in the plate thickness direction, and the strain ε at the end portion in the plate thickness direction is expressed by the following Formula (2).

$$\begin{matrix}[ Math . 2 ] & \\ ε = \frac{D}{2 ρ} = D \frac{M}{2 EI} & ( 2 )\end{matrix}$$

The dimension of the deformation guide 202 can be set such that, for example, in a case where the upper limit value of the strain in the elastic region of the material constituting the leaf spring 201 is εₘₐₓ, a strain larger than a predetermined allowable strain according to the upper limit value εₘₐₓ is not generated in the leaf spring 201. The allowable strain can be set to, for example, a value obtained by dividing the safety factor n (>1) by the upper limit value εₘₐₓ of the strain. When the allowable strain is generated in the leaf spring 201, the bending moment applied to the leaf spring 201 takes an upper limit value Mₘₐₓ, and the curvature radius of the leaf spring 201 takes a lower limit value ρₘᵢₙ. Therefore, the following Formula (3) indicating the relationship between the upper limit value εₘₐₓ of strain and the upper limit value Mₘₐₓ Of bending moment is derived from Formula (2).

$$\begin{matrix}[ Math . 3 ] & \\ \frac{{ε}_{max}}{n} = D \frac{{M}_{max}}{2 EI} & ( 3 )\end{matrix}$$

In addition, the following Formula (4) is derived by modifying Formula (3).

$$\begin{matrix}[ Math . 4 ] & \\ {M}_{max} = \frac{2 EI}{D} \frac{{ε}_{max}}{n} & ( 4 )\end{matrix}$$

In addition, the following Formula (5) indicating the relationship between the lower limit value ρₘᵢₙ of the curvature radius and the upper limit value Mₘₐₓ of the bending moment is derived from Formula (1).

$$\begin{matrix}[ Math . 5 ] & \\ {ρ}_{m i n} = \frac{EI}{{M}_{max}} & ( 5 )\end{matrix}$$

In addition, by substituting Formula (4) into Formula (5), the following Formula (6) indicating the relationship between the upper limit value εₘₐₓ of the strain and the lower limit value ρₘᵢₙ of the curvature radius is derived.

$$\begin{matrix}[ Math . 6 ] & \\ {ρ}_{m i n} = n \frac{D}{2 {ε}_{max}} & ( 6 )\end{matrix}$$

For example, as illustrated in FIGS. 6 and 10, the deformation guide 202 has a semicircular cross-sectional shape and extends in the width direction of the leaf spring 201. Specifically, the radius of the deformation guide 202 can be set to a lower limit value ρₘᵢₙ of the curvature radius calculated on the basis of Formula (6). Here, the bending moment applied to the leaf spring 201 by the load P is maximized at the one end portion 201a cantilevered among the respective positions of the leaf spring 201 as described later. Therefore, the curvature radius of the leaf spring 201 is minimum at the one end portion 201a of each position of the leaf spring 201.

In a case where the load P is small to such an extent that the bending moment applied to the one end portion 201a of the leaf spring 201 is equal to or less than the upper limit value Mₘₐₓ, the curvature radius at the one end portion 201a is equal to or greater than the lower limit value ρₘᵢₙ. Therefore, in such a case, as illustrated in FIG. 6, the portion on the one end portion 201a side of the leaf spring 201 does not abut on the outer peripheral portion of the deformation guide 202. Here, considering a process in which the load P increases from a relatively small value, when the bending moment applied to the one end portion 201a of the leaf spring 201 reaches the upper limit value Mₘₐₓ, the curvature radius at the one end portion 201a reaches the lower limit value ρₘᵢₙ. In a case where the load P further increases, the portion on the one end portion 201a side of the leaf spring 201 abuts on the outer peripheral portion of the deformation guide 202 in a state where the curvature radius is maintained at the lower limit value ρₘᵢₙ. As a result, the portion of the leaf spring 201 on the one end portion 201a side is supported by the deformation guide 202 on the bending direction side. Therefore, it is possible to prevent deformation of the leaf spring 201 in which the curvature radius is smaller than the lower limit value ρₘᵢₙ. Therefore, plastic deformation of the leaf spring 201 can be prevented.

As described above, in a case where the load P is equal to or less than the predetermined value, the portion of the leaf spring 201 on the one end portion 201a side does not abut on the outer peripheral portion of the deformation guide 202. On the other hand, in a case where the load P is larger than the predetermined value, the portion of the leaf spring 201 on the one end portion 201a side abuts on the outer peripheral portion of the deformation guide 202 and is supported on the bending direction side by the deformation guide 202. The predetermined value can correspond to, for example, the value of the load P when the bending moment applied to the one end portion 201a of the leaf spring 201 reaches the upper limit value Mₘₐₓ in the process of increasing the load P from a relatively small value.

(Deformation of Leaf Spring in Case where Applied Load is Equal to or Less than Predetermined Value)

Next, deformation of the leaf spring 201 in a case where the applied load P is equal to or less than a predetermined value will be described with reference to FIGS. 6 to 9. In a case where the load P is equal to or less than the predetermined value, the portion of the leaf spring 201 on the one end portion 201a side does not abut on the deformation guide 202. FIG. 6 illustrates a state of deformation of the leaf spring 201 in a state where the portion of the leaf spring 201 on the one end portion 201a side does not abut on the deformation guide 202.

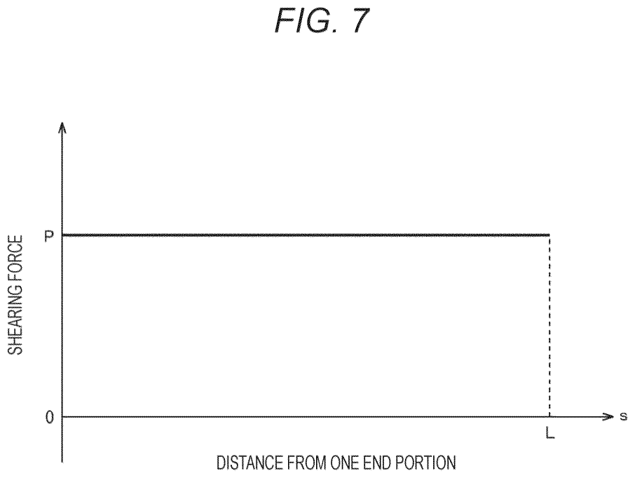

FIG. 7 is a shearing force diagram called a sheer force diagram (SFD) of the leaf spring 201 in the state illustrated in FIG. 6. The SFD in FIG. 7 illustrates the relationship between the distance s of the leaf spring 201 from the one end portion 201a in the longitudinal direction and the applied shearing force. As illustrated in FIG. 7, in a case where the applied load P is equal to or less than the predetermined value, the shearing force applied to the leaf spring 201 has the same value at the position corresponding to each distance s. Specifically, the shearing force applied to the leaf spring 201 has a value equal to the load P at a position corresponding to each distance s.

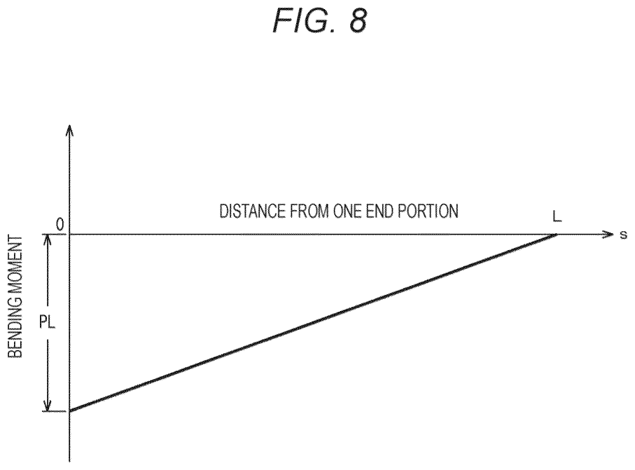

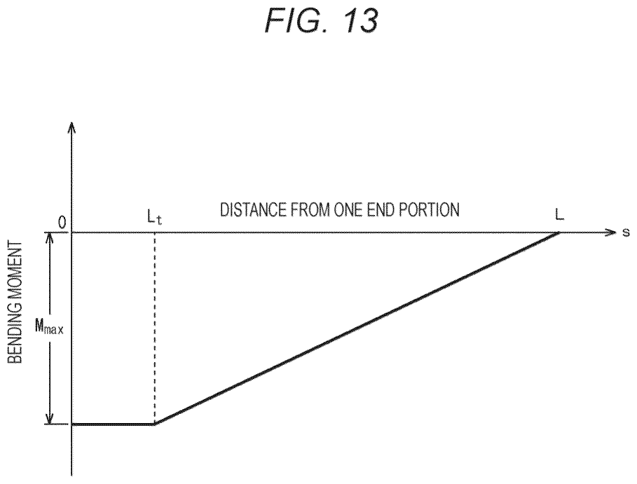

FIG. 8 is a bending moment diagram called a bending moment diagram (BMD) for the leaf spring 201 in the state illustrated in FIG. 6. In BMD of FIG. 8, the relationship between the distance s in the longitudinal direction from the one end portion 201a of the leaf spring 201 and the bending moment to be applied is illustrated. Note that, in the leaf spring 201, the change rate of the bending moment in the longitudinal direction coincides with the shearing force. Therefore, the bending moment represented by BMD in FIG. 8 and the shearing force represented by SFD in FIG. 7 have such a relationship. As illustrated in FIG. 8, in a case where the applied load P is equal to or less than the predetermined value, the absolute value of the bending moment applied to the leaf spring 201 increases from the other end portion 201b side toward the one end portion 201a side. Note that, in the BMDs of FIGS. 8 and 13, the value of the bending moment is expressed in consideration of the positive and negative directions, but in the following description, the value of the bending moment is expressed as an absolute value without considering the positive and negative directions.

Here, the bending moment M at each distance s of the leaf spring 201 is expressed by the following Formula (7) on the basis of the balance of the moments in the virtual cross section corresponding to each distance s.

$$\begin{matrix}[ Math . 7 ] & \\ M = P ( L - s ) & ( 7 )\end{matrix}$$

According to Formula (7), as indicated by BMD in FIG. 8, the absolute value of the bending moment applied to the leaf spring 201 takes 0 at the other end portion 201b and takes PL at the one end portion 201a. As described above, the bending moment applied to the leaf spring 201 is maximized at the one end portion 201a cantilevered among the respective positions of the leaf spring 201. Hereinafter, such a bending moment at the one end portion 201a is also referred to as maxM (s).

In addition, the curvature radius of the leaf spring 201 is minimum at the one end portion 201a of each position of the leaf spring 201. Hereinafter, the curvature radius at such one end portion 201a is also referred to as minρ (s). In a case where the load P is equal to or less than the predetermined value, as described above, the curvature radius minρ (s) at the one end portion 201a is equal to or greater than the lower limit value ρₘᵢₙ, and thus the following Formula (8) is established.

$$\begin{matrix}[ Math . 8 ] & \\ {ρ}_{m i n} \leq min ρ ( s ) & ( 8 )\end{matrix}$$

In addition, in a case where the load P is equal to or less than the predetermined value, as described above, the bending moment maxM(s) at the one end portion 201a is equal to or less than the upper limit value Mₘₐₓ. Furthermore, according to Formula (7), the bending moment maxM(s) at the one end portion 201a is PL. Therefore, the following Formula (9) is established.

$$\begin{matrix}[ Math . 9 ] & \\ {M}_{max} \geq max M ( s ) = P L & ( 9 )\end{matrix}$$

Here, the following Formula (10) is derived by modifying Formula (5).

$$\begin{matrix}[ Math . 10 ] & \\ {M}_{max} = \frac{EI}{{ρ}_{m i n}} & ( 10 )\end{matrix}$$

The following Formula (11) is derived by substituting Formula (10) into Formula (9) for arrangement.

$$\begin{matrix}[ Math . 11 ] & \\ \frac{EI}{L \cdot {ρ}_{m i n}} \geq P & ( 11 )\end{matrix}$$

According to Formula (11), in a case where the load P is equal to or less than EI/L·ρₘᵢₙ, the curvature radius minρ (s) at the one end portion 201a is equal to or greater than the lower limit value ρₘᵢₙ, so that the portion of the leaf spring 201 on the one end portion 201a side does not abut on the deformation guide 202. Therefore, in a case where the load P is equal to or less than EI/L·ρₘᵢₙ, the leaf spring 201 is in the state illustrated in FIG. 6.

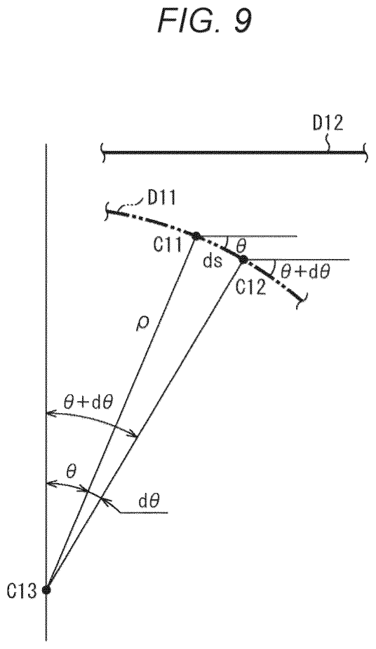

Here, the relationship between the deflection angle θ of the leaf spring 201 and the distance s from the one end portion 201a in the longitudinal direction will be described. FIG. 9 is an explanatory diagram for explaining the relationship between the deflection angle θ and the distance s. FIG. 9 schematically illustrates a deflection curve D11 indicating the neutral axis of the leaf spring 201 in a case where the bending deformation occurs and a straight line D12 indicating the neutral axis of the leaf spring 201 in a case where the bending deformation does not occur. As illustrated in FIG. 9, on the deflection curve D11, deflection angles for a point C11 and a point C12 located at minute distances ds from each other are θ and θ+dθ, respectively. Note that the deflection angle at each point on the deflection curve D11 is an angle formed by a tangent line of the deflection curve D11 at each point and the straight line D12. In addition, the curvature radius of the arc between the point C11 and the point C12 is defined as ρ, and the center of curvature is defined as a point C13.

In this case, as illustrated in FIG. 9, an angle formed by a straight line connecting the point C13 and the point C11 and a straight line orthogonal to the straight line D12 is θ. In addition, an angle formed by a straight line connecting the point C13 and the point C12 and a straight line orthogonal to the straight line D12 is θ+dθ. Therefore, the angle formed by the straight line connecting the point C13 and the point C11 and the straight line connecting the point C13 and the point C12 is de. Here, since de is a minute angle, the minute distance ds corresponding to the length of the arc between the point C11 and the point C12 is obtained by multiplying de, which is an angle formed by a straight line connecting the point C13 and the point C11 and a straight line connecting the point C13 and the point C12, by the curvature radius p. Therefore, the following Formula (12) is established.

$$\begin{matrix}[ Math . 12 ] & \\ ρ d θ = d s & ( 12 )\end{matrix}$$

In addition, the following Formula (13) is derived by solving Formulas (1) and (12) simultaneously and erasing ρ.

$$\begin{matrix}[ Math . 13 ] & \\ \frac{d θ}{d s} = \frac{M}{EI} & ( 13 )\end{matrix}$$

Here, since the other end portion deflection angle θ_{L}, which is the deflection angle at the other end portion 201b, corresponds to a value obtained by integrating the deflection angle θ from 0 to L with respect to the distance s, the following Formula (14) is established.

$$[ Math . 14 ]$$

$${θ}_{L} = {\int }_{s = 0}^{s = L} d θ = {\int }_{0}^{L} \frac{d θ}{ds} ds \qquad ( 14 )$$

Here, the following Formula (15) is derived by substituting Formula (13) into Formula (14).

$$[ Math . 15 ]$$

$${θ}_{L} = \frac{1}{EI} {\int }_{0}^{L} Mds \qquad ( 15 )$$

Here, the following Formula (16) is derived by substituting Formula (7) into Formula (15) for arrangement.

$$[ Math . 16 ]$$

$${θ}_{L} = \frac{1}{EI} {\int }_{0}^{L} P ( L - s ) ds = \frac{P}{EI} \frac{{L}^{2}}{2} \qquad ( 16 )$$

As described above, in a case where the load P is equal to or less than EI/L·ρₘᵢₙ, the portion of the leaf spring 201 on the one end portion 201a side does not abut on the deformation guide 202. In such a case, the other end portion deflection angle θ_{L}, which is the deflection angle at the other end portion 201b, is expressed by Formula (16).

(Deformation of Leaf Spring in Case where Applied Load is Larger than Predetermined Value)

Next, deformation of the leaf spring 201 in a case where the applied load P is larger than a predetermined value will be described with reference to FIGS. 10 to 13. In a case where the load P is larger than the predetermined value, the portion of the leaf spring 201 on the one end portion 201a side abuts on the deformation guide 202. FIG. 10 illustrates a state of deformation of the leaf spring 201 in a state where a portion of the leaf spring 201 on the one end portion 201a side abuts on the deformation guide 202.

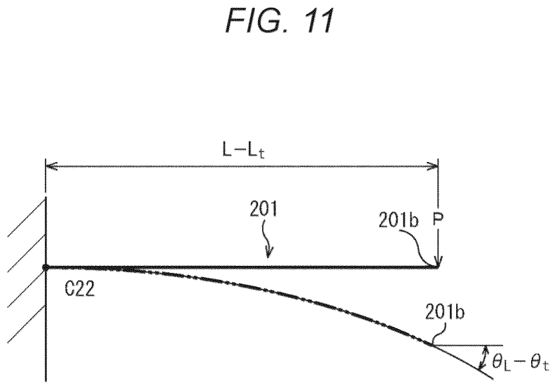

As illustrated in FIG. 10, on the one end portion 201a side of the leaf spring 201, a portion where the distance s from the one end portion 201a in the longitudinal direction is between 0 and Lₜ is a portion that abuts on the deformation guide 202. In addition, as illustrated in FIG. 10, in the deflection curve indicated by the two-dot chain line, a point located at a distance Lₜ from the one end portion 201a in the longitudinal direction is defined as a point C22. Specifically, the point C22 corresponds to a point on the neutral axis of the leaf spring 201 for the end portion on the opposite side to the one end portion 201a in the portion of the leaf spring 201 that abuts on the deformation guide 202. In this case, as illustrated in FIG. 11, a portion of the leaf spring 201 closer to the other end portion 201b side than the point C22 can correspond to a virtual leaf spring in which the point C22 is cantilevered.

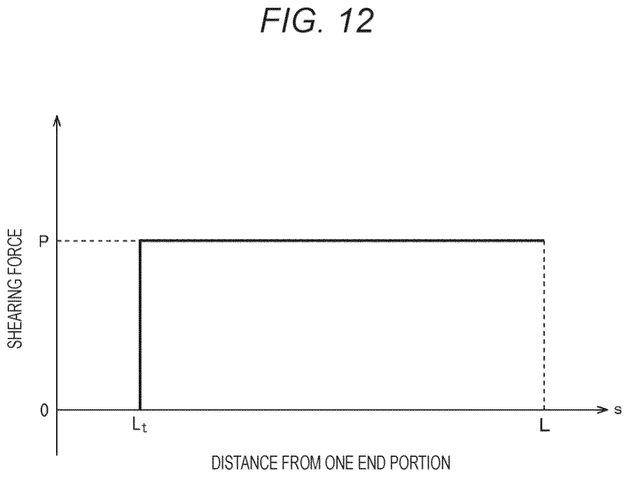

FIG. 12 illustrates the SFD of the leaf spring 201 in the state illustrated in FIG. 10. As illustrated in FIG. 12, in a case where the applied load P is larger than the predetermined value, the shearing force applied to the leaf spring 201 is 0 in the portion on the one end portion 201a side. On the one end portion 201a side of the leaf spring 201, a portion where the distance s is between 0 and Lₜ is supported by the deformation guide 202, and thus, the shearing force at the portion becomes 0. Therefore, the shearing force applied to the leaf spring 201 is specifically 0 in a portion where the distance s is between 0 and Lₜ, and is equal to the load P in a portion where the distance s is between Lₜ and L.

FIG. 13 illustrates BMDs of the leaf spring 201 in the state illustrated in FIG. 10. Note that, in the leaf spring 201, as described above, the change rate of the bending moment in the longitudinal direction coincides with the shearing force. Therefore, the bending moment represented by BMD in FIG. 13 and the shearing force represented by SFD in FIG. 12 have such a relationship. As illustrated in FIG. 13, the absolute value of the bending moment applied to the leaf spring 201 is the upper limit value Mₘₐₓ in the portion on the one end portion 201a side. Specifically, on the one end portion 201a side of the leaf spring 201, the curvature radius in the portion where the distance s is from 0 to Lₜ is the lower limit value ρₘᵢₙ, and thus, the absolute value of the bending moment is the upper limit value Mₘₐₓ in the portion. Therefore, the absolute value of the bending moment applied to the leaf spring 201 increases from the side of the other end portion 201b toward the side of the one end portion 201a in the portion where distance s is from Lₜ to L, and becomes the upper limit value Mₘₐₓ in the portion where distance s is from 0 to Lₜ.

Here, the upper limit value Mₘₐₓ is expressed by the following Formula (17) on the basis of the balance of the moments in the virtual cross section at the position where the distance s is Lₜ.

$$[ Math . 17 ]$$

$${M}_{max} = {\int }_{{L}_{t}}^{L} Pds = P ( L - {L}_{t} ) \qquad ( 17 )$$

Furthermore, the following Formula (18) is derived by modifying Formula (17).

$$[ Math . 18 ]$$

$$L - {L}_{t} = \frac{{M}_{max}}{P} \qquad ( 18 )$$

Here, as illustrated in FIG. 10, the center point of the semicircle representing the cross section of the deformation guide 202 is defined as a point C23, a point on the neutral axis of the leaf spring 201 with respect to the one end portion 201a is defined as a point C21, and an angle formed by a straight line connecting the point C23 and the point C21 and a straight line connecting the point C23 and the point C22 is defined as θₜ. Here, since θₜ is a minute angle, Lₜ corresponding to the length of the arc between the point C21 and the point C22 is obtained by multiplying θₜ, which is an angle formed by a straight line connecting the point C23 and the point C21 and a straight line connecting the point C23 and the point C22, by a lower limit value ρₘᵢₙ, which is a radius of the deformation guide 202. Therefore, the following Formula (19) is established.

$$[ Math . 19 ]$$

$${θ}_{t} = \frac{{L}_{t}}{{ρ}_{min}} \qquad ( 19 )$$

Here, the deflection angle at the point C22 of the leaf spring 201 is θₜ as illustrated in FIG. 10. Therefore, the virtual leaf spring in which the point C22 illustrated in FIG. 11 is cantilevered extends in a direction inclined by et in the bending direction with respect to the longitudinal direction of the leaf spring 201 in a case where the bending deformation as the virtual leaf spring does not occur. Therefore, as illustrated in FIG. 11, the deflection angle at the other end portion 201b of the virtual leaf spring corresponds to a value obtained by subtracting et from the other end portion deflection angle θ_{L} of the leaf spring 201 illustrated in FIG. 10.

Here, the deflection angle (θ_{L}−θₜ) at the other end portion 201b for the virtual leaf spring can be expressed by a Formula obtained by converting the integration interval for the distance s on the right side in Formula (15) into an interval from Lₜ to L, and thus the following Formula (20) is established.

$$[ Math . 20 ]$$

$${θ}_{L} - {θ}_{t} = \frac{1}{EI} {\int }_{{L}_{t}}^{L} Mds \qquad ( 20 )$$

In addition, the following Formula (21) is derived by substituting Formula (7) into Formula (20) for arrangement.

$$[ Math . 21 ]$$

$${θ}_{L} - {θ}_{t} = \frac{1}{EI} {\int }_{{L}_{t}}^{L} P ( L - s ) ds = \frac{1}{EI} \frac{{P ( L - {L}_{t} )}^{2}}{2} \qquad ( 21 )$$

Furthermore, the following Formula (22) is derived by substituting Formula (17) into Formula (21).

$$[ Math . 22 ]$$

$${θ}_{L} - {θ}_{t} = \frac{1}{EI} {M}_{max} \frac{L - {L}_{t}}{2} \qquad ( 22 )$$

In addition, the following Formula (23) is derived by solving Formulas (5) and (22) simultaneously and erasing the upper limit value Mₘₐₓ.

$$[ Math . 23 ]$$

$${θ}_{L} - {θ}_{t} = \frac{1}{{ρ}_{min}} \frac{L - {L}_{t}}{2} \qquad ( 23 )$$

In addition, the following Formula (24) is derived by substituting Formula (19) into Formula (23) for arrangement.

$$[ Math . 24 ]$$

$${θ}_{L} = \frac{1}{{ρ}_{min}} \frac{L + {L}_{t}}{2} \qquad ( 24 )$$

In addition, the following Formula (25) is derived by solving Formulas (18) and (24) simultaneously and erasing Lₜ.

$$[ Math . 25 ]$$

$${θ}_{L} = \frac{1}{{ρ}_{min}} \frac{2 L - {M}_{max} / P}{2} \qquad ( 25 )$$

In addition, the following Formula (26) is derived by solving Formulas (5) and (25) simultaneously and erasing the upper limit value Mₘₐₓ.

$$[ Math . 26 ]$$

$${θ}_{L} = \frac{L}{{ρ}_{min}} - \frac{1}{2 {ρ}_{min}^{2}} \frac{EI}{P} \qquad ( 26 )$$

As described above, in a case where the load P is larger than EI/L·ρₘᵢₙ, the portion of the leaf spring 201 on the one end portion 201a side abuts on the deformation guide 202. In such a case, the other end portion deflection angle θ_{L}, which is the deflection angle at the other end portion 201b, is expressed by Formula (26).

### [2-2. Mechanical Characteristics of Leaf Spring]

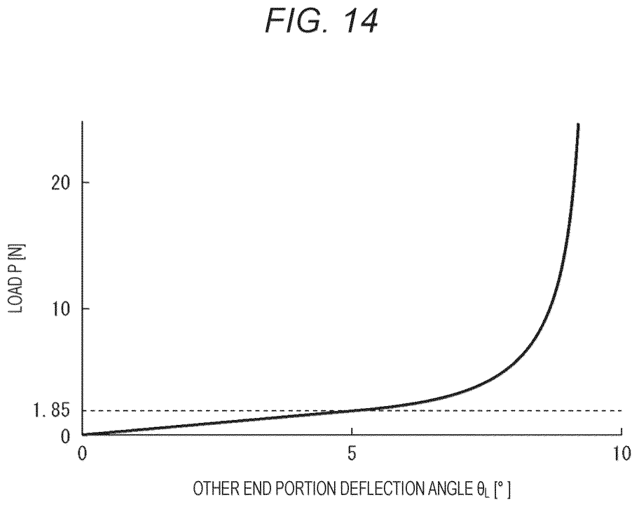

Next, mechanical characteristics of the leaf spring 201 will be described with reference to FIGS. 14 to 17. Hereinafter, as an example, a case where the length L in the longitudinal direction is set to 30 [mm], the plate thickness D is set to 0.6 [mm], the width W is set to 3 [mm], the Young's modulus E is set to 200 [GPa], the upper limit value εₘₐₓ of the strain is set to 2.5×10−3, and the safety factor n is set to 1.5 for each specification of the leaf spring 201 will be described.

In a case where each specification of leaf spring 201 is set as described above, second moment of area I of the leaf spring 201 is expressed by the following Formula (27).

$$[ Math . 27 ]$$

$$I = \frac{{WD}^{3}}{12} = 0.05 [ {mm}^{4} ] \qquad ( 27 )$$

In addition, the lower limit value ρₘᵢₙ of the curvature radius is expressed by the following Formula (28).

$$[ Math . 28 ]$$

$${ρ}_{min} = n \frac{D}{2 {ε}_{max}} = 180 [ mm ] \qquad ( 28 )$$

Therefore, the radius of the deformation guide 202 is set to 180 [mm], which is the lower limit value ρₘᵢₙ illustrated in Formula (28).

In a process in which the load P increases from a relatively small value, a predetermined value corresponding to the load P at the time of switching from a state in which a part of the leaf spring 201 does not abut on the deformation guide 202 to a state in which a part of the leaf spring 201 abuts on the deformation guide 202 is EI/L·ρₘᵢₙ. In a case where each specification of the leaf spring 201 is set as described above, the predetermined value is 1.85 [N].

FIG. 14 is an explanatory diagram illustrating an example of a relationship between the other end portion deflection angle θ_{L} of the leaf spring 201 and the load P applied to the leaf spring 201. FIG. 14 illustrates the relationship between the other end portion deflection angle θ_{L} defined by Formulas (16) and (26) and the load P. Specifically, the corresponding other end portion deflection angle θ_{L} can be calculated for each of the loads P of 1.85 [N] or less by using Formula (16). In addition, the corresponding other end portion deflection angle θ_{L} can be calculated for each of the loads P larger than 1.85 [N] by using Formula (26). FIG. 14 illustrates a result calculated in this manner.

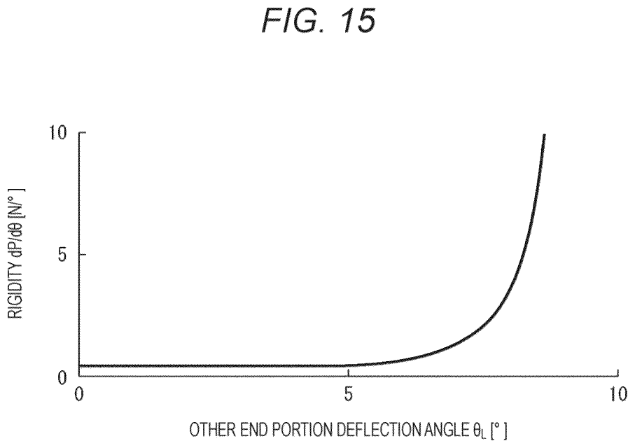

FIG. 15 is an explanatory diagram illustrating an example of the relationship between the other end portion deflection angle θ_{L} of the leaf spring 201 and the rigidity. The rigidity of the leaf spring 201 indicates the degree of difficulty of deformation of the leaf spring 201 with respect to the load P, and can be calculated on the basis of the relationship between the other end portion deflection angle θ_{L} and the load P illustrated in FIG. 14. Specifically, the rigidity for each of the other end portion deflection angles θ_{L} can be calculated by differentiating the load P by the other end portion deflection angle θ_{L} on the basis of the relationship between the other end portion deflection angle θ_{L} and the load P illustrated in FIG. 14. FIG. 15 illustrates a result calculated in this manner.

If the deformation guide 202 is not provided for the leaf spring 201, the leaf spring 201 can be deformed such that the curvature radius at the one end portion 201a becomes smaller than the lower limit value ρₘᵢₙ in a case where the load P exceeds 1.85 [N]. As a result, plastic deformation may occur in the leaf spring 201.

On the other hand, in the system illustrated in FIGS. 6 and 10, the deformation guide 202 is provided with respect to the leaf spring 201, so that the portion on the one end portion 201a side of the leaf spring 201 is supported on the bending direction side by the deformation guide 202 in a case where the load P exceeds 1.85 [N]. As a result, even in a case where the load P exceeds 1.85 [N], it is possible to prevent deformation of the leaf spring 201 in which the curvature radius becomes smaller than the lower limit value ρₘᵢₙ.

As described above, in a case where the load P is relatively large, a state in which the rigidity of the leaf spring 201 is high can be secured. FIG. 15 illustrates that the rigidity of the leaf spring 201 is relatively high in a case where the other end portion deflection angle θ_{L} correlated with the load P is relatively large. Specifically, in a case where the load P and the other end portion deflection angle θ_{L} are relatively large, the rigidity of the leaf spring 201 increases as the load P and the other end portion deflection angle θ_{L} increase. Therefore, as illustrated in FIG. 14, even in a case where the load P exceeds 1.85 [N], the leaf spring 201 can be elastically deformed by the displacement amount corresponding to each load P. Therefore, plastic deformation of the leaf spring 201 can be prevented.

Here, the load change rate of the leaf spring 201 will be described. The load change rate indicates the degree of change in the load P in a case where the other end portion deflection angle θ_{L} varies. Specifically, the load change rate is a change rate of the load P before and after the variation in a case where the other end portion deflection angle θ_{L} varies by 0.1°. Therefore, the load change rate is related to the measurement accuracy in the case of measuring the load P applied to the leaf spring 201 using the measurement value of the other end portion deflection angle θ_{L} as the displacement amount of the leaf spring 201. Specifically, the lower the load change rate, the higher the measurement accuracy. On the other hand, the higher the load change rate, the lower the measurement accuracy.

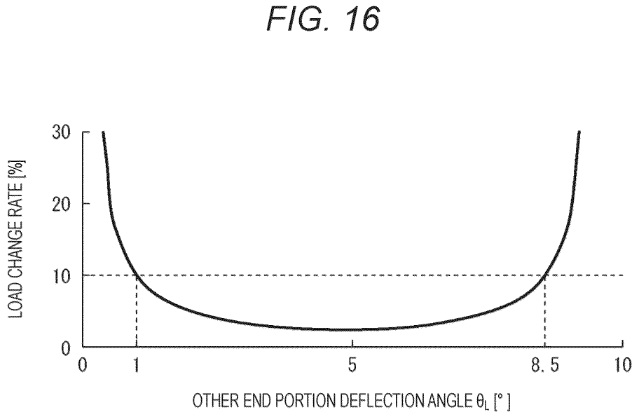

FIG. 16 is an explanatory diagram illustrating an example of the relationship between the other end portion deflection angle θ_{L} of the leaf spring 201 and the load change rate. The load change rate for each of the other end portion deflection angles θ_{L} can be calculated on the basis of the relationship between the other end portion deflection angle θ_{L} and the load P illustrated in FIG. 14. FIG. 16 illustrates a result calculated in this manner.

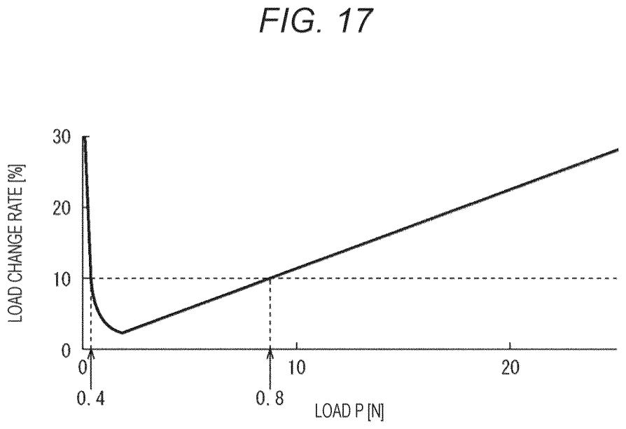

FIG. 17 is an explanatory diagram illustrating an example of the relationship between the load P applied to the leaf spring 201 and the load change rate. The load change rate for each of the loads P can be calculated on the basis of the relationship between the other end portion deflection angle θ_{L} and the load P illustrated in FIG. 14. FIG. 17 illustrates a result calculated in this manner.

In the system illustrated in FIGS. 6 and 10, in a case where the load P is 1.85 [N] or less, the portion on the one end portion 201a side of the leaf spring 201 does not abut on the outer peripheral surface of the deformation guide 202, and thus is not supported by the deformation guide 202. Thus, in a case where the load P is relatively small, a state in which the rigidity of the leaf spring 201 is low can be secured. FIG. 15 illustrates that the rigidity of the leaf spring 201 is relatively low in a case where the other end portion deflection angle θ_{L} correlated with the load P is relatively small.

Here, the lower the rigidity of the leaf spring 201, the lower the load change rate. In the system illustrated in FIGS. 6 and 10, in a case where the load P and the other end portion deflection angle θ_{L} are relatively small, the rigidity of the leaf spring 201 becomes relatively low, so that the load change rate becomes relatively low. FIG. 16 illustrates that the load change rate is relatively low in a case where the other end portion deflection angle θ_{L} correlated with the load P is relatively small. In addition, FIG. 17 illustrates that the load change rate is relatively low in a case where the load P is relatively small. Note that, since the load P is 0 [N] when the other end portion deflection angle θ_{L} is 0 [°], the load change rate can take a relatively large value when the load P and the other end portion deflection angle θ_{L} take values in the vicinity of 0 as illustrated in FIGS. 16 and 17.

As described above, in a case where the load P is relatively small, the load change rate is relatively low. Therefore, it is possible to ensure a state in which the measurement accuracy is high in a case where the load P applied to the leaf spring 201 is measured using the measurement value of the other end portion deflection angle θ_{L} as the displacement amount of the leaf spring 201.

The mechanical characteristics of the leaf spring 201 illustrated in FIGS. 16 and 17 define the relationship between the load change rate and the other end portion deflection angle θ_{L} and the relationship between the load change rate and the load P, respectively. Specifically, according to FIG. 16, the range of the other end portion deflection angle θ_{L} corresponding to the range in which the load change rate is less than 10% is a range from 1 [°] to 8.5 [°]. In addition, according to FIG. 17, the range of the load P corresponding to the range in which the load change rate is less than 10% is a range from 0.4 [N] to 8.8 [N]. Here, the mechanical characteristics of the leaf spring 201 depends on a set value of each specification of the leaf spring 201. Therefore, the range of the other end portion deflection angle θ_{L} and the load P corresponding to the range of the load change rate corresponding to the desired measurement accuracy can be appropriately set by appropriately setting each specification of the leaf spring 201. The mechanical characteristics of the leaf spring 201 also depends on the cross-sectional shape of the deformation guide 202. Therefore, the mechanical characteristics of the leaf spring 201 can be appropriately set by appropriately setting the cross-sectional shape of the deformation guide 202.

### 3. First Embodiment of Actuator

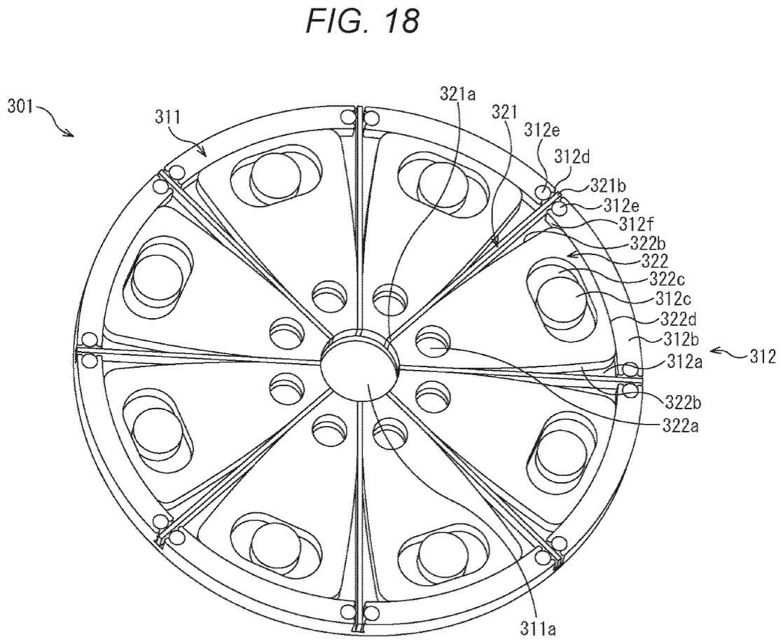

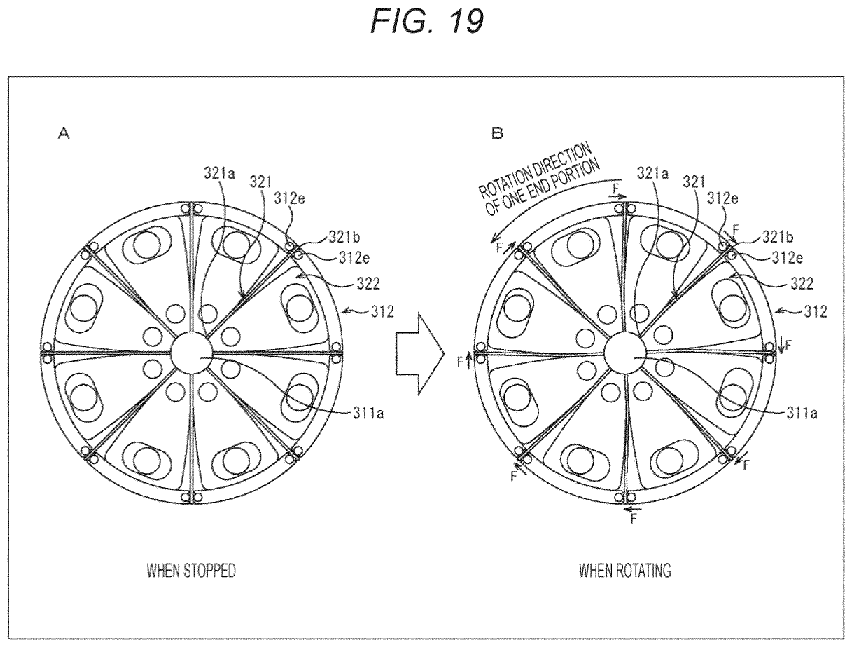

Next, an actuator 301, which is a first embodiment of an actuator applicable to the prosthesis 101 described above with reference to FIGS. 1 to 5, will be described with reference to FIGS. 18 and 19. The actuator 301 is an actuator similar to the actuator disclosed in Patent Document 1 described above.

FIG. 18 is a schematic diagram illustrating a part of the actuator 301 according to the first embodiment. A of FIG. 19 is a schematic diagram illustrating an example of a state of the actuator 301 in a case where a one end portion 321a of a leaf spring 321 is relatively stopped with respect to a rotating body 312. B of FIG. 19 is a schematic diagram illustrating an example of a state of the actuator 301 in a case where the one end portion 321a of the leaf spring 321 is relatively rotated with respect to the rotating body 312.

The actuator 301 includes a torque sensor 311 and the rotating body 312. The torque sensor 311 includes the leaf spring 321 for transmitting torque and a support member 322 for supporting a part of the leaf spring 321. The rotating body 312 is a member connected to the target object on the output side, and can constitute, for example, a part of the main body portion 121 of the thigh-side member 111 of the prosthesis 101. As illustrated in FIG. 19, the one end portion 321a of the leaf spring 321 is configured to be relatively rotatable with respect to the rotating body 312, so that torque is transmitted to the rotating body 312 via the leaf spring 321.

The actuator 301 transmits, for example, torque output from the drive motor 151. Specifically, the torque output from the drive motor 151 is input to the leaf spring 321 of the torque sensor 311. Then, the torque is transmitted to the rotating body 312 by the leaf spring 321. As described above, the leaf spring 321 transmits the torque output from the drive motor 151 to the rotating body 312. The rotating body 312 is connected to a target object on the output side, and can be configured such that the torque output from the actuator 301 is transmitted to the target object.

Specifically, in a case where the actuator 301 is applied to the prosthesis 101, the rotating body 312 is rotatably provided in synchronization with the main body portion 121 of the thigh-side member 111 as the target object on the output side. Therefore, the torque is transmitted to the rotating body 312 via the leaf spring 321, so that the main body portion 121 of the thigh-side member 111 including the rotating body 312 as a component rotates relative to the knee joint 122 and the lower-leg-side member 112.

The one end portion 321a of the leaf spring 321 is cantilevered. Specifically, the one end portion 321a of the leaf spring 321 is fixed to the output shaft 154 of the gear box 153 to be cantilevered. As illustrated in FIG. 18, the torque sensor 311 has, for example, a substantially disk shape, and a communication hole portion 311a communicating from one surface to the other surface along the central axis is provided in the central portion of the torque sensor 311. The one end portion 321a of the leaf spring 321 is fixed to the output shaft 154, for example, in a state where the output shaft 154 of the gear box 153 is inserted into the communication hole portion 311a. As a result, the one end portion 321a of the leaf spring 321 is rotatable integrally with the output shaft 154 of the gear box 153.

The rotation axis of the output shaft 154 of the gear box 153 may substantially coincide with the central axis of the communication hole portion 311a. In this case, the one end portion 321a of the leaf spring 321 is rotatable about the central axis of the communication hole portion 311a. In addition, the output shaft 154 of the gear box 153 is rotatable in synchronization with the output shaft 152 of the drive motor 151. Therefore, the one end portion 321a of the leaf spring 321 is rotatable in synchronization with the output shaft 152 of the drive motor 151.

For example, a plurality of leaf springs 321 is provided at intervals along the rotation direction of the one end portion 321a. Specifically, eight leaf springs 321 are provided at equal intervals along the rotation direction of the one end portion 321a. More specifically, as illustrated in FIG. 18, each of the eight leaf springs 321 extends along the radial direction of the communication hole portion 311a and is provided at equal intervals along the circumferential direction. The leaf spring 321 is disposed such that the width direction of the leaf spring 321 substantially coincides with the axial direction of the communication hole portion 311a. In other words, the leaf spring 321 is disposed such that the plate thickness direction of the leaf spring 321 substantially coincides with the circumferential direction of the communication hole portion 311a.

The support member 322 is a member for supporting a part of the leaf spring 321. For example, as illustrated in FIG. 18, the support member 322 has a flat plate shape, and is disposed such that a width direction of the support member 322 and a plate thickness direction of the leaf spring 321 substantially coincide with each other. Further, the plate thickness of the support member 322 and the width of the leaf spring 321 may substantially coincide with each other. The support member 322 has an opposing surface 322b facing the leaf spring 321 in the plate thickness direction of the leaf spring 321, and the opposing surface 322b abuts on a part of the leaf spring 321 in a case where the torque transmitted by the leaf spring 321 is larger than a predetermined value as described later.

For example, a plurality of support members 322 is provided at intervals along the rotation direction of the one end portion 321a of the leaf spring 321, and is located between two leaf springs 321 adjacent to each other in the rotation direction of the one end portion 321a. Specifically, in the torque sensor 311, as illustrated in FIG. 18, the leaf springs 321 and the support members 322 are alternately disposed along the circumferential direction of the torque sensor 311. As illustrated in FIG. 18, the communication hole portion 311a can be formed by the portion on the center side of the torque sensor 311 for each of the leaf spring 321 and the support member 322 disposed in this manner.

The relative movement of the support member 322 and the one end portion 321a of the leaf spring 321 is restricted. Accordingly, the support member 322 is rotatable integrally with the one end portion 321a of the leaf spring 321. Specifically, a through hole 322a for attaching the output shaft 154 of the gear box 153 to the torque sensor 311 is formed on the communication hole portion 311a side of the support member 322. For example, a member such as a screw for connecting the support member 322 to the connection member 156 can be inserted into the through hole 322a. The support member 322 and the one end portion 321a of the leaf spring 321 are fixed to the output shaft 154 of the gear box 153 by using the through hole 322a.

The rotating body 312 is rotatable relative to the one end portion 321a of the leaf spring 321. For example, as illustrated in FIG. 18, the rotating body 312 includes a base portion 312a facing the plurality of support members 322 in the plate thickness direction of the support member 322 and having an annular disk shape, and a protruding portion 312b provided along the outer peripheral portion of the base portion 312a and protruding from the base portion 312a toward the support member 322 side.

The base portion 312a is provided with a projecting portion 312c protruding toward the support member 322 side at a position corresponding to each support member 322. A through hole 322c extending along the circumferential direction of the communication hole portion 311a is formed in an outer peripheral side portion of the torque sensor 311 for each of the support members 322, and the projecting portion 312c is inserted into the through hole 322c. The through hole 322c and the projecting portion 312c can have a function as a guide that defines the rotation direction of the rotating body 312. Further, the side surface 322d on the outer peripheral side of the torque sensor 311 for each of the support members 322 abuts on the inner peripheral surface 312f of the protruding portion 312b. The side surface 322d of the support member 322 and the inner peripheral surface 312f of the protruding portion 312b may also have a function as a guide that defines the rotation direction of the rotating body 312.

The rotation direction of the rotating body 312 may substantially coincide with the circumferential direction of the communication hole portion 311a. In other words, the rotating body 312 may be rotatable about a rotation axis of the one end portion 321a of the leaf spring 321. As a result, since each member constituting the actuator 301 can be rotated around a common rotation axis, the actuator 301 can be more effectively downsized.

In the protruding portion 312b, a groove portion 312d is formed at a position corresponding to each leaf spring 321 along the radial direction of the communication hole portion 311a. The groove portion 312d has a width larger than the plate thickness of the leaf spring 321, and a portion of the leaf spring 321 on the other end portion 321b side is inserted and fitted into the groove portion 312d. Specifically, pins 312e extending in the width direction of the leaf spring 321 are provided on surfaces of the groove portion 312d facing each other in the width direction, and a portion of the leaf spring 321 on the other end portion 321b side is inserted and fitted between the pair of pins 312e. Thus, the leaf spring 321 is supported in the plate thickness direction by the pair of pins 312e. Accordingly, the other end portion 321b of the leaf spring 321 is rotatable integrally with the rotating body 312.

As described above, the leaf spring 321 transmits the torque output from the drive motor 151 to the rotating body 312. In addition, the one end portion 321a of the leaf spring 321 is rotatable in synchronization with the output shaft 152 of the drive motor 151. In addition, the other end portion 321b of the leaf spring 321 is rotatable integrally with the rotating body 312. In addition, the one end portion 321a of the leaf spring 321 is configured to be relatively rotatable with respect to the rotating body 312. Therefore, in a case where the torque is input from the drive motor 151 to the one end portion 321a of the leaf spring 321, as illustrated in FIG. 19, the one end portion 321a of the leaf spring 321 relatively rotates with respect to the other end portion 321b, whereby the leaf spring 321 is bent and deformed in the plate thickness direction. Then, a load corresponding to the restoring force of the leaf spring 321 is applied from the other end portion 321b of the leaf spring 321 to the pin 312e of the rotating body 312. As a result, torque is transmitted to the rotating body 312 via the leaf spring 321. As described above, the leaf spring 321 is bendable and deformable in the plate thickness direction according to the torque by transmitting the torque.

Here, as described above, the one end portion 321a of the leaf spring 321 is cantilevered. In addition, as illustrated in FIG. 19, in a case where the leaf spring 321 is bent and deformed in the plate thickness direction according to the torque by transmitting the torque, reaction force F from the pin 312e of the rotating body 312 is applied as a load to the other end portion 321b of the leaf spring 321 in the plate thickness direction. The reaction force F has a magnitude corresponding to the degree of bending deformation of the leaf spring 321. Therefore, considering that the one end portion 321a of the leaf spring 321 is fixed, the deformation of the leaf spring 321 can be considered similar to the deformation of the leaf spring 201 when the load P is applied in the plate thickness direction to the other end portion 201b of the leaf spring 201 in which the one end portion 201a is cantilevered, described with reference to FIGS. 6 to 17. Note that the reaction force F as the load applied from the pin 312e of the rotating body 312 to the other end portion 321b of the leaf spring 321 corresponds to the load P applied to the other end portion 201b of the leaf spring 201 described with reference to FIGS. 6 to 17. In addition, the reaction force F has a correlation with the torque transmitted by the leaf spring 321.

The support member 322 according to the present embodiment supports a part of the leaf spring 321 on the bending direction side in a case where the torque transmitted by the leaf spring 321 is larger than a predetermined value. Specifically, in a case where the torque transmitted by the leaf spring 321 is larger than a predetermined value, the opposing surface 322b of the support member 322 abuts on a part of the leaf spring 321. As a result, a part of the leaf spring 321 is supported by the support member 322 on the bending direction side. The predetermined value can correspond to a value of the torque when the bending moment applied to the one end portion 321a of the leaf spring 321 reaches the bending moment corresponding to the predetermined allowable strain corresponding to the upper limit value of the strain in the elastic region of the material constituting leaf spring 321 in the process of increasing the transmitted torque from a relatively small value.

Here, as described above, the support member 322 is rotatable integrally with the one end portion 321a of the leaf spring 321. Therefore, the relationship between the support member 322 and the leaf spring 321 according to the present embodiment can be considered similarly to the relationship between the deformation guide 202 and the leaf spring 201 in the system described with reference to FIGS. 6 to 17. For example, the support member 322 may be provided close to a portion of the one end portion 321a side of the leaf spring 321 in the plate thickness direction of the leaf spring 321, and support the portion of the one end portion 321a side of the leaf spring 321 on the bending direction side in a case where the torque transmitted by the leaf spring 321 is larger than a predetermined value.

Further, the shape of the opposing surface 322b may be set on the basis of the lower limit value ρₘᵢₙ of the curvature radius expressed by Formula (6). Specifically, the cross-sectional curve of the opposing surface 322b in the cross section orthogonal to the width direction of the leaf spring 321 may be an arc whose curvature radius is a lower limit value ρₘᵢₙ. Further, the cross-sectional curve of the opposing surface 322b in the cross section orthogonal to the width direction of the leaf spring 321 may be a part of the deflection curve of the leaf spring 321 on the one end portion 321a side in a case where the transmitted torque is a predetermined value. Note that the lower limit value ρₘᵢₙ can be calculated on the basis of each specification of the leaf spring 321.

According to the present embodiment, since the support member 322 is provided with respect to the leaf spring 321, in a case where the torque transmitted by the leaf spring 321 is larger than a predetermined value, a part of the leaf spring 321 is supported on the bending direction side by the support member 322. As a result, even in a case where the transmitted torque exceeds the predetermined value, it is possible to prevent deformation of the leaf spring 321 in which the curvature radius becomes smaller than the lower limit value ρₘᵢₙ. As described above, in a case where the transmitted torque is relatively large, the high rigidity of the leaf spring 321 can be secured. Therefore, plastic deformation of the leaf spring 321 can be prevented. Therefore, the actuator 301 can be downsized while securing the strength of the leaf spring 321. Therefore, the device including the actuator 301 can be downsized.

In addition, according to the present embodiment, in a case where the torque transmitted by the leaf spring 321 is equal to or less than the predetermined value, a part of the leaf spring 321 does not abut on the opposing surface 322b of the support member 322, and thus is not supported by the support member 322. Accordingly, in a case where the transmitted torque is relatively small, it is possible to ensure a state where the rigidity of the leaf spring 321 is low. Here, the lower the rigidity of the leaf spring 321, the lower the load change rate indicating the degree of change in the reaction force F applied to the other end portion 321b in a case where the other end portion deflection angle θ_{L} varies. Therefore, in a case where the transmitted torque is relatively small, the load change rate of the leaf spring 321 is relatively low.

Here, the displacement amount of the leaf spring 321 can be detected by the displacement sensor 181 as described above. Specifically, the displacement sensor 181 can detect the other end portion deflection angle θ_{L}, which is the deflection angle at the other end portion 321b, as the displacement amount of the leaf spring 321. Then, the reaction force F applied to the other end portion 321b of the leaf spring 321 can be calculated by the control device 182 on the basis of the measurement value of the other end portion deflection angle θ_{L} corresponding to the detection result obtained by the displacement sensor 181. In addition, the lower the load change rate, the higher the measurement accuracy. Therefore, according to the present embodiment, in a case where the torque transmitted by the leaf spring 321 is relatively small, it is possible to secure a state in which the measurement accuracy is high in the case of measuring the reaction force F applied to the other end portion 321b of the leaf spring 321.

Further, the mechanical characteristics of the leaf spring 321 depends on a set value of each specification of the leaf spring 321. Specifically, the relationship between the load change rate and the reaction force F and the relationship between the load change rate and the other end portion deflection angle θ_{L} in the leaf spring 321 depend on setting values of specifications of the leaf spring 321. Therefore, by appropriately setting each specification of the leaf spring 321, the range of the other end portion deflection angle θ_{L} and the reaction force F corresponding to the range of the load change rate corresponding to the desired measurement accuracy can be appropriately set. Note that the length L, the plate thickness D, the width W, the Young's modulus E, the upper limit value εₘₐₓ of strain, and the safety factor n in the longitudinal direction of the leaf spring 321 can correspond to the specifications of the leaf spring 321. The mechanical characteristics of the leaf spring 321 also depends on the shape of the opposing surface 322b of the support member 322. Therefore, the mechanical characteristics of the leaf spring 321 can be appropriately set by appropriately setting the shape of the opposing surface 322b of the support member 322.

In addition, the actuator 301 includes the leaf spring 321, the support member 322, and the rotating body 312, and has a relatively simple configuration. Therefore, it is possible to more effectively reduce the size and weight of the device including the actuator 301.

The leaf spring 321 is constituted by, for example, spring steel. Further, each of the support member 322 and the rotating body 312 may be constituted by resin. Specifically, each of the support member 322 and the rotating body 312 may be constituted by nylon, polypropylene (PP), or the like. As described above, since the support member 322 or the rotating body 312 is constituted by resin, the weight of the device can be reduced.

As described above, for example, a plurality of leaf springs 321 is provided at intervals along the rotation direction of the one end portion 321a. As a result, the bending moment applied to each leaf spring 321 can be reduced as compared with a case where the number of leaf springs 321 provided in the actuator 301 is one. Therefore, it is possible to more effectively prevent plastic deformation from occurring in the leaf spring 321. Further, by appropriately setting the number of leaf springs 321 provided in the actuator 301, the mechanical characteristics of each leaf spring 321 can be appropriately set.

In addition, as described above, for example, a plurality of support members 322 is provided at intervals along the rotation direction of the one end portion 321a of the leaf spring 321, and are located between two leaf springs 321 adjacent to each other in the rotation direction of the one end portion 321a. As a result, the plurality of leaf springs 321 and the plurality of support members 322 can be configured to be symmetrical with respect to a plane including their rotation axes. Therefore, even in a case where the direction of the torque transmitted by the actuator 301 is reversed, it is possible to obtain actions and effects similar to those before the reverse rotation with respect to the support of the leaf spring 321 by the support member 322.

### 4. Second Embodiment of Actuator

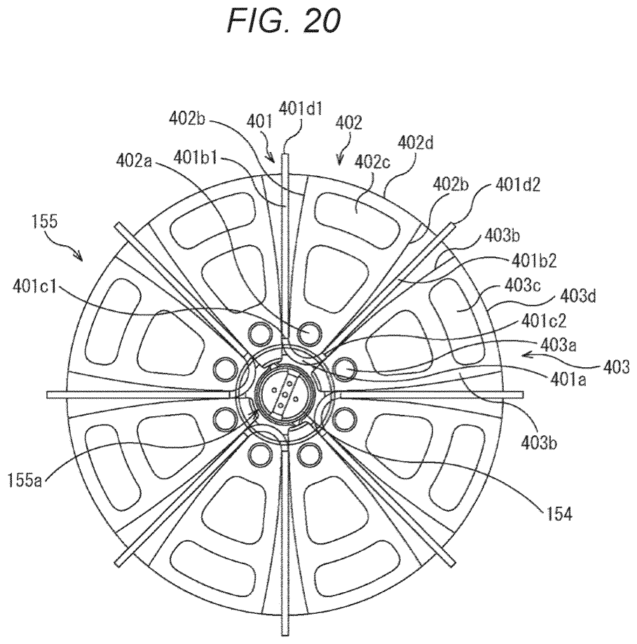

Next, a second embodiment of an actuator applicable to the prosthesis 101 described above with reference to FIGS. 1 to 5 will be described with reference to FIG. 20. Note that the second embodiment is different from the first embodiment in the configuration of the torque sensor. Therefore, here, a difference in the configuration of the torque sensor will be mainly described.

FIG. 20 is a schematic diagram illustrating an example of a configuration of the torque sensor 155 applicable to the actuator 301 described above with reference to FIGS. 18 and 19 and applicable to the prosthesis 101 described above with reference to FIGS. 1 to 5. Note that illustration of the rotating body 312 is omitted here.

As compared with the torque sensor 311 of FIG. 18, the torque sensor 155 includes four leaf springs 401 instead of the leaf springs 321, and includes four support members 402 and four support members 403 instead of the support members 322.

Similarly to the communication hole portion 311a of the torque sensor 311 of FIG. 18, the communication hole portion 155a is formed at the center of the torque sensor 155. The output shaft 154 of the gear box 153 is inserted into the communication hole portion 155a. The rotation axis of the output shaft 154 of the gear box 153 substantially coincides with the central axis of the communication hole portion 155a.

The leaf spring 401 has a shape in which a central portion in the extending direction of the plate-shaped member is bent in a U-shape in the plate thickness direction. That is, the tip of a bent portion 401a of the leaf spring 401 is bent so as to be rounded. An extending portion 401b1 and an extending portion 401b2 linearly extend from both ends of a bent portion 401a. The extending portion 401b1 and the extending portion 401b2 are directed obliquely to each other.

The bent portion 401a of the leaf spring 401 faces the communication hole portion 155a, and a plurality of leaf springs 401 is provided at intervals along the rotation direction of the bent portion 401a (=the circumferential direction of the communication hole portion 155a). Specifically, four leaf springs 401 are provided at equal intervals along the rotation direction of the bent portion 401a. The leaf spring 401 is disposed such that the width direction of the leaf spring 401 substantially coincides with the axial direction of the communication hole portion 155a. In other words, the leaf spring 401 is disposed such that the plate thickness direction of the leaf spring 401 substantially coincides with the circumferential direction of the communication hole portion 155a. The extending portion 401b1 and the extending portion 401b2 of each leaf spring 401 extend along the radial direction of the communication hole portion 155a and are provided at equal intervals along the circumferential direction.

The support member 402 and the support member 403 have a flat plate shape substantially similar to that of the support member 322 in FIG. 18. The support member 402 and the support member 403 are disposed such that the width direction of the support member 402 and the support member 403 substantially coincides with the plate thickness direction of the leaf spring 401. The plate thicknesses of the support member 402 and the support member 403 and the width of the leaf spring 401 may substantially coincide with each other.

The bent portion 401a of each leaf spring 401 is fitted to the end portion of the support member 402 on the communication hole portion 155a side. As a result, the position of the leaf spring 401 is fixed and stabilized. Further, the support member 402 is disposed between the extending portion 401b1 and the extending portion 401b2 of the same leaf spring 401.

Further, the support member 403 is disposed between the two leaf springs 401 adjacent in the rotation direction of the bent portion 401a. Thus, the leaf spring 401 and the support member 402 are sandwiched between two adjacent support members 403. In other words, the support member 403 is sandwiched between two adjacent sets of the leaf springs 401 and the support member 402. In addition, a one end portion 401c1, which is an end on the bent portion 401a side of the extending portion 401b1 of the leaf spring 401, is sandwiched and fixed between the support member 402 and the support member 403. A one end portion 401c2, which is an end on the bent portion 401a side of the extending portion 401b2 of the leaf spring 401, is sandwiched and fixed between the support member 402 and the support member 403.

A through hole 402a for attaching the output shaft 154 of the gear box 153 to the torque sensor 155 via the connection member 156 is formed on the communication hole portion 155a side of each support member 402. For example, a member such as a screw for connecting the support member 402 to the connection member 156 can be inserted into the through hole 402a.

A through hole 403a for attaching the output shaft 154 of the gear box 153 to the torque sensor 155 via the connection member 156 is formed on the communication hole portion 155a side of each support member 403. For example, a member such as a screw for connecting the support member 402 to the connection member 156 can be inserted into the through hole 403a.

By attaching the support members 402 and the support members 403 to the connection member 156 through the through holes 402a and 403a, the support members 402 and the support members 403 are fixed to the output shaft 154 of the gear box 153. Thus, the leaf springs 401 fixed by the support members 402 and the support members 403 are fixed to the output shaft 154. As a result, the one end portion 401cl of the extending portion 401b1 and the one end portion 401c2 of the extending portion 401b2 of each leaf spring 401 are cantilevered with respect to the output shaft 154.

A portion on the other end portion 401d1 side of the extending portion 401b1 of each leaf spring 401 and a portion on the other end portion 401d2 side of the extending portion 401b2 are inserted and fitted into the groove portion 312d of the rotating body 312, and are inserted and fitted between the pair of pins 312e. As a result, a portion on the other end portion 401d1 side of the extending portion 401b1 of each leaf spring 401 and a portion on the other end portion 401d2 side of the extending portion 401b2 are supported by the pair of pins 312e in the plate thickness direction. Therefore, a portion on the other end portion 401d1 side of the extending portion 401b1 of each leaf spring 401 and the other end portion 401d2 of the extending portion 401b2 are integrally rotatable with the rotating body 312.

The support member 402 has an opposing surface 402b facing the extending portion 401b1 or the extending portion 401b2 of the leaf spring 401 in the plate thickness direction of the extending portion 401b1 or the extending portion 401b2. Similarly to the opposing surface 322b of the support member 322 in FIG. 18, in a case where the torque transmitted by the extending portion 401b1 or the extending portion 401b2 is larger than a predetermined value, the opposing surface 402b abuts on a part of the extending portion 401b1 or the extending portion 401b2 and supports the extending portion 401b1 or a part of the extending portion 401b2.

The support member 403 has an opposing surface 403b facing the extending portion 401b1 or the extending portion 401b2 of the leaf spring 401 in the plate thickness direction of the extending portion 401b1 or the extending portion 401b2. Similarly to the opposing surface 322b of the support member 322 in FIG. 18, in a case where the torque transmitted by the extending portion 401b1 or the extending portion 401b2 is larger than a predetermined value, the opposing surface 403b abuts on a part of the extending portion 401b1 or the extending portion 401b2 and supports the extending portion 401b1 or a part of the extending portion 401b2. Note that, in a case where the torque transmitted clockwise by the extending portion 401b1 is larger than a predetermined value, the extending portion 401b1 is supported by the opposing surface 403b of the support member 403 adjacent in the counterclockwise direction. In a case where the torque transmitted counterclockwise by the extending portion 401b1 is larger than a predetermined value, the extending portion 401b1 is supported by the opposing surface 402b of the support member 402 adjacent in the clockwise direction. In a case where the torque transmitted clockwise by the extending portion 401b2 is larger than a predetermined value, the extending portion 401b2 is supported by the opposing surface 402b of the support member 402 adjacent in the counterclockwise direction. In a case where the torque transmitted counterclockwise by the extending portion 401b2 is larger than a predetermined value, the extending portion 401b2 is supported by the opposing surface 403b of the support member 403 adjacent in the clockwise direction.

A through hole 402c extending along the circumferential direction of the communication hole portion 155a is formed in an outer peripheral side portion of the torque sensor 155 of each support member 402. Similarly to the through hole 322c of the support member 322 in FIG. 18, the projecting portion 312c of the rotating body 312 is inserted into the through hole 402c. The through hole 402c and the projecting portion 312c can have a function as a guide that defines the rotation direction of the rotating body 312. Further, the side surface 402d on the outer peripheral side of the torque sensor 155 of each support member 402 abuts on the inner peripheral surface 312f of the protruding portion 312b of the rotating body 312. The side surface 402d of the support member 402 and the inner peripheral surface 312f of the protruding portion 312b may also have a function as a guide that defines the rotation direction of the rotating body 312.

A through hole 403c extending along the circumferential direction of the communication hole portion 311a is formed in an outer peripheral side portion of the torque sensor 311 of each support member 403. Similarly to the through hole 322c of the support member 322 in FIG. 18, the projecting portion 312c of the rotating body 312 is inserted into the through hole 403c. The through hole 403c and the projecting portion 312c can have a function as a guide that defines the rotation direction of the rotating body 312. Further, the side surface 403d on the outer peripheral side of the torque sensor 155 of each support member 403 abuts on the inner peripheral surface 312f of the protruding portion 312b of the rotating body 312. The side surface 403d of the support member 403 and the inner peripheral surface 312f of the protruding portion 312b may also have a function as a guide that defines the rotation direction of the rotating body 312.

The torque sensor 155 configured as described above realizes functions similar to those of the torque sensor 311 in FIG. 18, and exhibits similar actions and effects. That is, the extending portion 401b1 and the extending portion 401b2 of each leaf spring 401 of the torque sensor 155 realize functions similar to those of the leaf spring 321 of FIG. 18 and exhibit similar actions and effects. Each support member 402 and the support member 403 of the torque sensor 155 realize functions similar to the support member 322 of the torque sensor 311 in FIG. 18, and exhibit actions and effects similar to the support member 322.

For example, in a case where the torque transmitted by the extending portion 401b1 and the extending portion 401b2 of the leaf spring 401 is larger than a predetermined value, a part of the extending portion 401b1 and the extending portion 401b2 is supported on the bending direction side by the support member 402 and the support member 403. Therefore, in a case where the transmitted torque is relatively large, it is possible to ensure a state in which the rigidity of the leaf spring 401 is high. Therefore, plastic deformation of the leaf spring 401 can be prevented. Therefore, the actuator 301 can be downsized while securing the strength of the leaf spring 401. Therefore, the device including the actuator 301 can be downsized.

Further, for example, in a case where the torque transmitted by the extending portion 401b1 and the extending portion 401b2 of the leaf spring 401 is equal to or less than a predetermined value, a part of the extending portion 401b1 and the extending portion 401b2 does not abut on the opposing surface 402b of the support member 402 and the opposing surface 403b of the support member 403, and is not supported by the support member 402 and the support member 403. As a result, in a case where the transmitted torque is relatively small, it is possible to ensure a state in which the rigidity of the extending portion 401b1 and the extending portion 401b2 of the leaf spring 401 is low. Therefore, in a case where the transmitted torque is relatively small, the load change rates of the extending portion 401b1 and the extending portion 401b2 are relatively low. In addition, the lower the load change rate, the higher the measurement accuracy in the case of measuring the reaction force F applied to the one end portion 401cl of the extending portion 401b1 and the one end portion 401c2 of the extending portion 401b2 using the measurement value of the other end portion deflection angle θ_{L} as the displacement amount of the extending portion 401b1 and the extending portion 401b2.

In addition, in the torque sensor 155, as described above, the bent portion 401a of the leaf spring 401 is fitted to the end portion of the support member 402 on the communication hole portion 155a side. This facilitates positioning of the leaf spring 401, and improves stability of the position of the leaf spring 401. For example, even in a case where the torque sensor 155 rotates in the circumferential direction and torque is input to the leaf spring 401, positional displacement of the leaf spring 401 hardly occurs. Further, generation of frictional sound between the leaf spring 401 and the support member 402 and the support member 403 due to positional displacement of the leaf spring 401 is suppressed.

On the other hand, in the torque sensor 311, the one end portion 321a side of the leaf spring 321 is only sandwiched between the two support members 322. Therefore, in the torque sensor 311, in order to stabilize the position of the leaf spring 321, for example, a member for fixing the one end portion 321a of each leaf spring 321 at the outer peripheral portion of the communication hole portion 311a is required. In addition, for example, in a case where the torque sensor 311 rotates in the circumferential direction and torque is input to the leaf spring 321, there is a possibility that positional displacement of the leaf spring 321 occurs. Furthermore, frictional sound may be generated between the leaf spring 321 and the support member 322 due to positional displacement of the leaf spring 321.

In addition, the torque sensor 155 can reduce the number of leaf springs and reduce the number of assembling steps as compared with the torque sensor 311.

### 5. Modifications

Note that, in the above description, an example has been described in which the support member 402 and the support member 403 support the portion on the one end portion 401cl side of the extending portion 401b1 on the bending direction side in a case where the torque transmitted by the extending portion 401b1 of the leaf spring 401 is larger than the predetermined value, but the supported portion is not limited to such an example. For example, a portion of the extending portion 401b1 closer to the other end portion 401d1 side than the one end portion 401cl may be supported by the support member 402 and the support member 403. Further, a plurality of portions in the extending portion 401b1 may be supported by the support member 402 and the support member 403.

Similarly, for example, a portion of the extending portion 401b2 of the leaf spring 401 closer to the other end portion 401d2 side than the one end portion 401c2 may be supported by the support member 402 and the support member 403. Further, a plurality of portions in the extending portion 401b2 may be supported by the support member 402 and the support member 403.

In the above description, an example has been described in which the torque is transmitted to the main body portion 121 of the thigh-side member 111 by the actuator 301, and the thigh-side member 111 is relatively rotated with respect to the lower-leg-side member 112. On the other hand, for example, the torque can be transmitted to the knee joint 122 of the thigh-side member 111 by the actuator 301, and the lower-leg-side member 112 connected to the knee joint 122 can be relatively rotated with respect to the thigh-side member 111.

The actuator according to the present technology can be applied to other than the above-described prosthesis 101. For example, the actuator according to the present technology can be applied to joint portions of various robots. Furthermore, for example, the actuator according to the present technology can be applied to various devices including a rotation unit.

### 6. Others

The preferred embodiments of the present disclosure have been described above in detail with reference to the accompanying drawings, but the technical scope of the present disclosure is not limited to such examples. It is obvious that those with ordinary skill in the technical field of the present disclosure can conceive various modifications or corrections within the scope of the technical idea recited in the claims, and it is naturally understood that these modifications or corrections also fall within the technical scope of the present disclosure.

Further, the effects disclosed in the present specification are merely illustrative or exemplary, but are not restrictive. That is, the technology according to the present disclosure may achieve other effects obvious to those skilled in the art from the description in the present specification, in addition to or instead of the effects described above.

Note that the following configurations also belong to the technical scope of the present disclosure.
(1)
   An actuator including:
   a leaf spring that includes a bent portion bent in a U shape and a first extending portion extending from the bent portion, a first one end portion that is an end portion of the first extending portion on a side of the bent portion being cantilevered, the first extending portion being bendable and deformable in a plate thickness direction according to torque by transmitting the torque; and
   a first support member that supports a part of the first extending portion on a bending direction side in a case where torque transmitted in a first direction by the first extending portion is larger than a predetermined value.
(2)
   The actuator according to (1), in which
   the leaf spring further includes a second extending portion extending obliquely from the bent portion with respect to the first extending portion,
   a second one end portion which is an end portion of the second extending portion on the side of the bent portion is cantilevered,
   the second extending portion is bendable and deformable in the plate thickness direction according to the torque by transmitting the torque, and
   the first support member supports a part of the second extending portion on the bending direction side in a case where torque transmitted in a second direction opposite to the first direction by the second extending portion is larger than a predetermined value.
(3)
   The actuator according to (2), further including a second support member that supports a part of the first extending portion on the bending direction side in a case where the torque transmitted in the second direction by the first extending portion is larger than a predetermined value, or supports a part of the second extending portion on the bending direction side in a case where the torque transmitted in the first direction by the second extending portion is larger than a predetermined value.
(4)
   The actuator according to (3), in which
   a plurality of the leaf springs is provided at intervals along a rotation direction of the bent portion,
   the first support member is disposed between the first extending portion and the second extending portion of the same leaf spring, and
   the second support member is disposed between two adjacent leaf springs.
(5)
   The actuator according to (4), in which a communication hole portion is formed in a central portion where the leaf spring, the first support member, and the second support member are disposed along the rotation direction of the bent portion.
(6)
   The actuator according to (5), further including a first output shaft having a hollow structure that is inserted into the communication hole portion and transmits the torque to the first extending portion and the second extending portion.
(7)
   The actuator according to (6), further including:
   a drive motor;
   a second output shaft having a hollow structure and configured to output torque of the drive motor; and
   a gear that converts the torque transmitted by the second output shaft at a predetermined reduction ratio, in which
   the first output shaft transmits the torque output from the gear to the first extending portion and the second extending portion.
(8)
   The actuator according to (7), in which
   a rotation axis of the first output shaft and a rotation axis of the second output shaft substantially coincide with each other, and
   a hollow portion of the first output shaft and a hollow portion of the second output shaft overlap each other in an axial direction.
(9)
   The actuator according to any one of (2) to (8), in which the first support member includes:
   a first surface that supports a part of the first extending portion on the bending direction side in a case where the torque transmitted in the first direction by the first extending portion is larger than a predetermined value; and
   a second surface that supports a part of the second extending portion on the bending direction side in a case where the torque transmitted in the second direction by the second extending portion is larger than a predetermined value.
(10)
   The actuator according to any one of (2) to (9), in which the bent portion of the leaf spring is fitted to one end of the first support member.
(11)
   The actuator according to (2) or (3), in which a plurality of the leaf springs is provided at intervals along a rotation direction of the bent portion.
(12)
   The actuator according to (11), in which four leaf springs are provided at equal intervals along the rotation direction of the bent portion.
(13)
   The actuator according to (1) or (2), further including a second support member that supports a part of the first extending portion on the bending direction side in a case where torque transmitted in a second direction opposite to the first direction by the first extending portion is larger than a predetermined value.
(14)
   The actuator according to (13), in which the first support member and the second support member sandwich the first one end portion of the first extending portion.
(15)
   The actuator according to any one of (1) to (14), further including a rotating body that is relatively rotatable with respect to one end portion of the first extending portion, in which
   the first extending portion transmits torque output from a drive motor to the rotating body,
   one end portion of the first extending portion is rotatable in synchronization with an output shaft of the drive motor, and
   another end portion of the first extending portion is rotatable integrally with the rotating body.
(16)
   The actuator according to (15), in which the first support member is rotatable integrally with the one end portion of the first extending portion.
(17)
   The actuator according to (15) or (16), in which the rotating body is rotatable around a rotation axis of the one end portion of the first extending portion.
(18)
   A prosthesis including:
   a thigh-side member;
   a lower-leg-side member; and
   an actuator that connects the thigh-side member and the lower-leg-side member, and transmits torque to one of the thigh-side member and the lower-leg-side member to rotate one of the thigh-side member and the lower-leg-side member relative to the other, in which
   the actuator includes:
      a leaf spring including a bent portion bent in a U shape and an extending portion extending from the bent portion, one end portion which is an end portion of the extending portion on a side of the bent portion being cantilevered, the extending portion being bendable and deformable in a plate thickness direction according to torque by transmitting the torque; and
      a support member that supports a part of the extending portion on a bending direction side in a case where torque transmitted by the extending portion is larger than a predetermined value.

### REFERENCE SIGNS LIST

101 Prosthesis
111 Thigh-side member
112 Lower-leg-side member
121 Main body portion
121a Connection portion
122 Knee joint
122a Connection portion
122b Housing portion
131 Extending portion
132 Ground contact portion
151 Drive motor
152 Output shaft
152a Hollow portion
153 Gear box
154 Output shaft
155 Torque sensor
156 Connection member
157 to 162 Bearing
181 Displacement sensor
182 Control device
301 Actuator
312 Rotating body
312a Base portion
312b Protruding portion
312c Projecting portion
312d Groove portion
312e Pin
312f Inner peripheral surface
401 Leaf spring
401a Bent portion
401b1, 401b2 Extending portion
401c1, 401c2 One end portion
401d1, 401d2 Other end portion
402 Support member
402a Through hole
402b Opposing surface
402c Through hole
402d Side surface
403 Support member
403a Through hole
403b Opposing surface
403c Through hole
403d Side surface

1. An actuator comprising:
a leaf spring that includes a bent portion bent in a U shape and a first extending portion extending from the bent portion, a first one end portion that is an end portion of the first extending portion on a side of the bent portion being cantilevered, the first extending portion being bendable and deformable in a plate thickness direction according to torque by transmitting the torque; and
a first support member that supports a part of the first extending portion on a bending direction side in a case where torque transmitted in a first direction by the first extending portion is larger than a predetermined value.

2. The actuator according to claim 1, wherein
the leaf spring further includes a second extending portion extending obliquely from the bent portion with respect to the first extending portion,
a second one end portion which is an end portion of the second extending portion on the side of the bent portion is cantilevered,
the second extending portion is bendable and deformable in the plate thickness direction according to the torque by transmitting the torque, and
the first support member supports a part of the second extending portion on the bending direction side in a case where torque transmitted in a second direction opposite to the first direction by the second extending portion is larger than a predetermined value.

3. The actuator according to claim 2, further comprising a second support member that supports a part of the first extending portion on the bending direction side in a case where the torque transmitted in the second direction by the first extending portion is larger than a predetermined value, or supports a part of the second extending portion on the bending direction side in a case where the torque transmitted in the first direction by the second extending portion is larger than a predetermined value.

4. The actuator according to claim 3, wherein
a plurality of the leaf springs is provided at intervals along a rotation direction of the bent portion,
the first support member is disposed between the first extending portion and the second extending portion of the same leaf spring, and
the second support member is disposed between two adjacent leaf springs.

5. The actuator according to claim 4, wherein a communication hole portion is formed in a central portion where the leaf spring, the first support member, and the second support member are disposed along the rotation direction of the bent portion.

6. The actuator according to claim 5, further comprising a first output shaft having a hollow structure that is inserted into the communication hole portion and transmits the torque to the first extending portion and the second extending portion.

7. The actuator according to claim 6, further comprising:
a drive motor;
a second output shaft having a hollow structure and configured to output torque of the drive motor; and
a gear that converts the torque transmitted by the second output shaft at a predetermined reduction ratio, wherein
the first output shaft transmits the torque output from the gear to the first extending portion and the second extending portion.

8. The actuator according to claim 7, wherein
a rotation axis of the first output shaft and a rotation axis of the second output shaft substantially coincide with each other, and
a hollow portion of the first output shaft and a hollow portion of the second output shaft overlap each other in an axial direction.

9. The actuator according to claim 2, wherein the first support member includes:
a first surface that supports a part of the first extending portion on the bending direction side in a case where the torque transmitted in the first direction by the first extending portion is larger than a predetermined value; and
a second surface that supports a part of the second extending portion on the bending direction side in a case where the torque transmitted in the second direction by the second extending portion is larger than a predetermined value.

10. The actuator according to claim 2, wherein the bent portion of the leaf spring is fitted to one end of the first support member.

11. The actuator according to claim 2, wherein a plurality of the leaf springs is provided at intervals along a rotation direction of the bent portion.

12. The actuator according to claim 11, wherein four leaf springs are provided at equal intervals along the rotation direction of the bent portion.

13. The actuator according to claim 1, further comprising a second support member that supports a part of the first extending portion on the bending direction side in a case where torque transmitted in a second direction opposite to the first direction by the first extending portion is larger than a predetermined value.

14. The actuator according to claim 13, wherein the first support member and the second support member sandwich the first one end portion of the first extending portion.

15. The actuator according to claim 1, further comprising a rotating body that is relatively rotatable with respect to one end portion of the first extending portion, wherein
the first extending portion transmits torque output from a drive motor to the rotating body,
one end portion of the first extending portion is rotatable in synchronization with an output shaft of the drive motor, and
another end portion of the first extending portion is rotatable integrally with the rotating body.

16. The actuator according to claim 15, wherein the first support member is rotatable integrally with the one end portion of the first extending portion.

17. The actuator according to claim 15, wherein the rotating body is rotatable around a rotation axis of the one end portion of the first extending portion.

18. A prosthesis comprising:
a thigh-side member;
a lower-leg-side member; and
an actuator that connects the thigh-side member and the lower-leg-side member, and transmits torque to one of the thigh-side member and the lower-leg-side member to rotate one of the thigh-side member and the lower-leg-side member relative to the other, wherein
the actuator includes:
a leaf spring including a bent portion bent in a U shape and an extending portion extending from the bent portion, one end portion which is an end portion of the extending portion on a side of the bent portion being cantilevered, the extending portion being bendable and deformable in a plate thickness direction according to torque by transmitting the torque; and
a support member that supports a part of the extending portion on a bending direction side in a case where torque transmitted by the extending portion is larger than a predetermined value.
